(19) United States
(12) Patent Application Publication
(10) Pub. No.: US 2017/0182031 A1
(43) Pub. Date: Jun. 29, 2017

(54) REDUCING DRUG LIKING IN A SUBJECT

(71) Applicant: Purdue Pharma, Pickering (CA)

(72) Inventors: Joseph L. REIZ, Markham (CA); Kenneth J. MICHALKO, Whitby (CA)

(21) Appl. No.: 15/300,240

(22) PCT Filed: Mar. 27, 2015
(86) PCT No.: PCT/CA2015/000206

§ 371 Date: Sep. 28, 2016

Related U.S. Application Data
(60) Provisional application No. 61/967,851, filed on Mar. 28, 2014.

(30) Foreign Application Priority Data
Mar. 28, 2014 (CA) 2847.781

(51) Int. Cl. A61K 31/485; A61K 9/20; A61K 9/50; A61K 9/00; A61K 9/48

(52) U.S. Cl. CPC A61K 31/485; A61K 9/0053; A61K 9/4858; A61K 9/5042; A61K 9/4866; A61K 9/5078; A61K 9/5084; A61K 9/2018; A61K 9/2013

(57) ABSTRACT

There is described a method of reducing drug liking a subject comprising the step of administering to the subject an oral pharmaceutical composition comprising: (i) hydromorphone or a pharmaceutically acceptable salt thereof, and (ii) naloxone or a pharmaceutically acceptable salt thereof, wherein the oral pharmaceutical composition comprising (i) and (ii) in a weight ratio equal to or less than about 4:1. There is also described use of an oral pharmaceutical composition comprising: (i) hydromorphone or a pharmaceutically acceptable salt thereof, and (ii) naloxone or a pharmaceutically acceptable salt thereof, wherein the oral pharmaceutical composition comprising (i) and (ii) in a weight ratio equal to or less than about 4:1, for reducing drug liking in a subject. The present inventors have conducted clinical studies from which it can be concluded that that drug liking in opioid abusers can be reduced when the weight ratio of (i) and (ii) in an intravenous composition is equal to or less than about 4:1. Based on the these clinical studies, the present inventors have established a reasonable inference that similar results would be obtained in the case of oral pharmaceutical compositions having a corresponding weight ratio of (i) and (ii)—i.e., if such an oral composition were to be abused by extraction of (i) and (ii) therefrom, the resulting extraction composition would behave in a similar manner as the intravenous compositions used in the clinical studies reported herein.

### CROSS-REFERENCE TO RELATED APPLICATION

The present application claims the benefit under 35 U.S.C. §119(e) of provisional patent application Ser. 61/967,851, filed Mar. 28, 2014, the contents of which are hereby incorporated by reference.

### BACKGROUND OF THE INVENTION

Field of the Invention

The present invention relates to reducing drug liking in a subject, more particularly a subject taking hydromorphone or a pharmaceutically acceptable salt thereof.

Description of the Prior Art

Hydromorphone (HMO) is an efficacious and potent analgesic indicated for the treatment of severe pain.

However, HMO is also recognized to have a high risk for abuse and dependence, and has recently been associated with increasing rates of misuse and abuse. For example, emergency department visits associated with abuse or misuse of HMO have increased by ˜259% from an estimated 3,385 in 2004 to an estimated 12,142 in 2008 in the United States (Substance Abuse and Mental Health Services Administration [SAMHSA], 2010).

Although drug use via the intravenous (IV) route of administration occurs less frequently compared to oral and intranasal use (Hays et al., 2003), intravenous abuse can carry a much greater public health risk, including higher rates of overdose, relapse, and transmission of blood-borne diseases, ie, HIV and hepatitis (Health Canada, 2001; Hays et al., 2003; Bargagli et al., 2006; SAMHSA, 2010). Therefore, development of a formulations that deters intravenous abuse could have important public health benefits

One approach to abolish the subjective effects of an opioid is to concurrently administer an opioid antagonist. A balance needs to be established between maintaining therapeutic efficacy of the opioid when administered as indicated (i.e., oral) and abating its euphoric effects when the opioid is tampered with and taken in an unintended form, e.g., IV administration.

Due to its very low oral bioavailability (i.e., <3%), naloxone is an attractive choice as an antagonist, because it is unlikely to affect the analgesic properties of HMO when administered orally. In contrast, if a modified-release tablet containing both HMO and naloxone is crushed and dissolved for injection, the presence of naloxone in the solution should abate the euphoric effects of HMO. In opioid-dependent subjects this may precipitate withdrawal, thus, reducing the risk of abuse via this route. This approach as been used in prior oral formulations containing opioids such as oxycodone.

The Food and Drug Administration in the United States published in January 2013 a Draft Guidance document entitled: “Guidance for Industry Abuse-Deterrent Opioids—Evaluation and Labeling”. This document states that Visual Analogue Scales (VAS) is an instrument that may be used to assess drug abuse potential. VAS are used for drug liking, good effects, bad effects and other drug abuse-related effects. More particularly, the document states that, for the evaluation of the abuse potential of a potentially abuse-deterrent formulation, VAS for drug liking should be the primary measure as it appears to correlate most directly with potential for abuse.

Thus, it would be advantageous to have a formulation containing HMO which possessed improved abuse deterrence when assessed using VAS for drug liking (i.e., a formulation that conferred reduced drug liking in a subject such an abuser).

### SUMMARY OF THE INVENTION

It is an object of the present invention to obviate or mitigate at least one of the above-mentioned disadvantages of the prior art.

It is another object of the present invention to provide a novel approach for reducing drug liking a subject, particularly a subject taking hydromorphone a pharmaceutically acceptable salt thereof.

Accordingly, in one of its aspects, the present invention provides a method of reducing drug liking in a subject comprising the step of administering to the subject an oral pharmaceutical composition comprising: (i) hydromorphone or a pharmaceutically acceptable salt thereof, and (ii) naloxone or a pharmaceutically acceptable salt thereof, wherein the oral pharmaceutical composition comprising (i) and (ii) in a weight ratio equal to or less than about 4:1.

In another of its aspects, the present invention provides use of an oral pharmaceutical composition comprising: (i) hydromorphone or a pharmaceutically acceptable salt thereof, and (ii) naloxone or a pharmaceutically acceptable salt thereof, wherein the oral pharmaceutical composition comprising (i) and (ii) in a weight ratio equal to or less than about 4:1, for reducing drug liking in a subject.

The present invention relates to reducing drug liking in a subject. This may be achieved by administration of an oral pharmaceutical composition comprising: (i) hydromorphone or a pharmaceutically acceptable salt thereof, and (ii) naloxone or a pharmaceutically acceptable salt thereof, wherein the oral pharmaceutical composition comprising (i) and (ii) in a weight ratio equal to or less than about 4:1

A drug abuser will typically try to extract (i) from such an oral pharmaceutical composition using solvent extract, mechanical crushing etc. This results in extraction of composition containing both (i) and (ii). It is this extraction composition that the drug abuser will inject in an effort to obtain the high from (i). The present inventors have discovered that reduced drug liking by the drug abuser may be achieved at certain ratios of (i) and (ii) in the extraction composition (and thus in original oral pharmaceutical), notwithstanding the antagonist effects of (ii) on (i) when administered intravenously (vs. orally).

As described hereinbelow, the present inventors have conducted clinical studies from which it can be concluded that that drug liking in opioid abusers can be reduced when the weight ratio of (i) and (ii) in an intravenous composition is equal to or less than about 4:1. Based on the these clinical studies, the present inventors have established a reasonable inference that similar results would be obtained in the case of oral pharmaceutical compositions having a corresponding weight ratio of (i) and (ii)—i.e., if such an oral composition were to be abused by extraction of (i) and (ii) therefrom, the resulting extraction composition would behave in a similar manner as the intravenous compositions used in the clinical studies reported below.

### BRIEF DESCRIPTION OF THE DRAWINGS

Embodiments of the present invention will be described with reference to the accompanying drawings, wherein like reference numerals denote like parts, and in which:

FIGS. 1-6 illustrate results from the clinical study reported in Example 1; and

FIGS. 7-12 illustrate results from the clinical study reported in Example 2.

### DETAILED DESCRIPTION OF THE PREFERRED EMBODIMENTS

In one of its aspects, the present invention relates to a method of reducing drug liking a subject comprising the step of administering to the subject an oral pharmaceutical composition comprising: (i) hydromorphone or a pharmaceutically acceptable salt thereof, and (ii) naloxone or a pharmaceutically acceptable salt thereof, wherein the oral pharmaceutical composition comprising (i) and (ii) in a weight ratio equal to or less than about 4:1. In another of its aspects, the present invention relates to use of an oral pharmaceutical composition comprising: (i) hydromorphone or a pharmaceutically acceptable salt thereof, and (ii) naloxone or a pharmaceutically acceptable salt thereof, wherein the oral pharmaceutical composition comprising (i) and (ii) in a weight ratio equal to or less than about 4:1, for reducing drug liking in a subject. Preferred embodiments of this method or use may include any one or a combination of any two or more of any of the following features:
the oral pharmaceutical composition comprises (i) and (ii) in a weight ratio in the range of from about 4:1 to about 1:1;
the oral pharmaceutical composition comprises (i) and (ii) in a weight ratio in the range of from about 4:1 to about 1:1.5;
the oral pharmaceutical composition comprises (i) and (ii) in a weight ratio in the range of from about 3.5:1 to about 1:1.5;
the oral pharmaceutical composition comprises (i) and (ii) in a weight ratio in the range of from about 3:1 to about 1:1.5;
the oral pharmaceutical composition comprises (i) and (ii) in a weight ratio in the range of from about 2.5:1 to about 1:1.5;
the oral pharmaceutical composition comprises (i) and (ii) in a weight ratio of about 2:1;
the subject is a drug user;
the subject is an opiod drug user;
the subject is a recreational drug user;
the subject is a recreational opioid user;
the subject is a drug user who took an opioid for non-therapeutic purposes on at least 10 occasions during the 12 month period prior to the step of administering to the subject the oral pharmaceutical composition;
the subject is a dependent drug user;
the subject is an opioid-dependent drug user.
(i) is a pharmaceutically acceptable salt of hydromorphone;
(i) is hydromorphone hydrochloride;
(ii) is a pharmaceutically acceptable salt of naloxone;
(i) is naloxone hydrochloride;
the oral pharmaceutical composition comprises a prolonged release pharmaceutical composition;
the prolonged release pharmaceutical composition comprises a prolonged release pharmaceutical dosage form comprising a plurality of coated beads, each of the coated beads comprising:
   (a) a granule;
   (b) a first layer coated on the granule, the first layer comprising: (i) hydromorphone or a pharmaceutically acceptable salt thereof, (ii) naloxone or a pharmaceutically acceptable salt thereof, (iii) an antioxidant compound, and (iii) a chelating compound; and
   (c) a second layer coated on the first layer, the second layer comprising a prolonged release agent;
the antioxidant compound comprises sodium metabisulfite;
the chelating agent comprises ethylenedinitrotetraacetic acid;
the chelating agent comprises ethylenedinitrotetraacetic acid disodium salt;
the prolonged release compound is selected from the group consisting of a hydrophobic polymer, a hydrophilic polymer, a protein-derived material, a gum, a substituted or unsubstituted hydrocarbon, a digestible carbohydrate, a fatty acid, a fatty alcohol, a glyceryl ester of a fatty acid, a natural oil, a synthetic oil, a natural wax, a synthetic wax and any mixture of two or more of any of these;
the prolonged release compound is selected from the group consisting of a cellulose ether, an acrylic based polymer, an acrylic based copolymer, a methacrylic based polymer, a methacrylic based copolymer, a fatty alcohol and any mixture of two or more of any of these;
the prolonged release compound is selected from the group consisting of a neutral acrylic based polymer, a neutral acrylic based copolymer, a neutral methacrylic based polymer, a neutral methacrylic based copolymer, a hydrophobic cellulose ether, a fatty alcohol and any mixture of two or more of any of these;
the prolonged release compound is ethyl cellulose;
the granule is selected from an uncoated microcrystalline cellulose granule and a mannitol-polyvinylpyrrolidone granule;
the granule further comprises:
   (d) a third layer coated on the second layer, the third layer comprising a moisture barrier agent.
the moisture barrier agent comprises a polyvinyl alcohol-polyethylene glycol graft copolymer;
the prolonged release composition is in the form of a capsule;
the capsule contains the plurality of coated beads;
the capsule is a hydroxypropyl methyl cellulose capsule;
the prolonged release pharmaceutical composition comprises a prolonged release pharmaceutical dosage form comprising a plurality of coated beads disposed in a hydroxypropyl methyl cellulose capsule, each of the coated beads comprising:
   (a) a granule;
   (b) a first layer coated on the granule, the first layer comprising: (i) hydromorphone hydrochloride, (ii) naloxone hydrochloride, (iii) an antioxidant compound, and (iii) a chelating compound, wherein (i) and (ii) are present in a weight ratio of about 2:1;
   (c) a second layer coated on the first layer, the second layer comprising ethyl cellulose; and
   (d) a third layer coated on the second layer, the third layer comprising a polyvinyl alcohol-polyethylene glycol graft copolymer.
the granule is an uncoated microcrystalline cellulose granule;
the granule is a mannitol-polyvinylpyrrolidone granule;
the prolonged release pharmaceutical composition comprises a prolonged release pharmaceutical dosage form in the form of a capsule containing coated beads derived from the following formulation:

| Ingredient | Amount per capsule (mg) |
|---|---|
| Hydromorphone hydrochloride | 3.00 |
| Naloxone hydrochloride dihydrate | 1.65 |
| Microcrystalline cellulose spheres | 44.72 |
| Hydroxypropyl methycellulose/ | 0.50 |
| polyethylene glycol | |
| Sodium metabisulfite | 0.05 |
| Disodium EDTA dihydrate | 0.05 |
| Aqueous ethylcellulose dispersion | 3.95 |
| Polyvinyl alcohol-polyethylene glycol | 1.41 |
| graft copolymer | |
| Water | q.s. (to 55 mg) |

the oral pharmaceutical composition comprises an immediate release pharmaceutical composition;
the immediate release pharmaceutical composition comprises a diluent;
the immediate release pharmaceutical composition comprises a colourant;
the immediate release pharmaceutical composition comprises a lubricant;
the immediate release pharmaceutical composition is derived from one of the following formulations:

| | Strength | | | |
|---|---|---|---|---|
| | 1/0.5 mg | 2/1 mg | 4/2 mg | 8/4 mg |
| Tablet Core Constituent | mg/tablet | mg/tablet | mg/tablet | mg/tablet |
| Hydromorphone | 1.00 | 2.00 | 4.00 | 8.00 |
| hydrochloride¹ | | | | |
| Naloxone hydrochloride | 0.55 | 1.10 | 2.20 | 4.40 |
| dihydrate² | | | | |
| Lactose anhydrous | 84.45 | 84.00 | 84.00 | 136.10 |
| DC Yellow #10 Lake | 0.30 | 0.40 | 0.30 | — |
| FD&C Blue #1 Lake | 0.04 | — | — | — |
| FD&C Red #30 Lake | — | 0.06 | — | — |
| Sodium stearyl fumarate | 1.36 | 1.34 | 1.40 | 2.30 |
| Total core | ~88 | ~89 | ~92 | ~151 |

¹calculated based on assay
²calculated based on assay and moisture content;

and/or
concurrently while treating pain in the subject.

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

Where an indefinite or definite article is used when referring to a singular noun, for example, “a”, “an” or “the”, this includes a plural of that noun unless something else is specifically stated.

In the context of the present invention the terms “about” or “approximately” denote an interval of accuracy that the person skilled in the art will understand to still ensure the technical effect of the feature in question. The term typically indicates deviation from the indicated numerical value off 10%, and preferably of ±5%.

The term “in vitro release” and its grammatical variations as well as similar expression refers to the release rate by which a pharmaceutically active agent, for example, hydromorphone HCl is released from the pharmaceutical composition when the in vitro release rate is tested by the paddle method according to the European Pharmacopeia as described in the Ph. Eur. 2.9.3 6^{th} edition. The paddle speed is typically set at 75 or 100 rpm in 500 ml or 900 ml simulated gastric fluid (SGF) dissolution medium with pH 1.2. Aliquots of the dissolution media are withdrawn at the respective time points and analysed by HPLC with a C18 column, eluted with 30 mM phosphate buffer in acetonitrile (70:70; pH 2.9) with a flow rate of 1.0 ml/min and detected at 220 nm. It is specifically indicated if in the context of the present invention in vitro release rates are determined using a different test method (such as SGF with 40% (v/v) of ethanol).

The amount of dissolution liquid and the rotational speed of the paddle apparatus may depend on the amount of active agent tested. For example, pharmaceutical compositions comprising up to 16 mg hydromorphone HCl may be tested at 75 rpm in 500 ml dissolution liquid while higher dosage strengths may be tested at 100 rpm in 900 ml dissolution liquid.

The term “Simulated Gastric Fluid, pH 1.2” refers to 0.1 N HCl, pH 1.2.

In the context of the present invention, the terms “immediate release” or “conventional release” refer to pharmaceutical compositions showing a release of the active substance(s) which is not deliberately modified by a special formulation design and/or manufacturing methods. For oral dosage forms this means that the dissolution profile of the active substance(s) depends essentially on its (theirs) intrinsic properties. Typically, the terms “immediate release” or “conventional release” refer to pharmaceutical compositions which release in vitro >75% (by weight) of the pharmaceutically active agent(s) at 45 min.

In the context of the present, the terms “prolonged release” and “controlled release” are used interchangeably and refer to pharmaceutical compositions showing a slower release of the active agent(s) than that of a conventional release pharmaceutical composition administered by the same route. Prolonged or controlled release is achieved by a special formulation design and/or manufacturing method. Typically, the terms “prolonged release” and “controlled release refer to pharmaceutical compositions which release in vitro ≦75% (by weight) of the pharmaceutically active agent at 45 min.

Prolonged release properties may be obtained by different means such as by a coating which is then designated as a prolonged release coating.

In order to obtain “prolonged or controlled release” properties, one typically uses materials which are known to prolong the release from a dosage form comprising such as a prolonged release coating. Typical examples of such “prolonged or controlled release materials” are hydrophobic polymers such as ethyl cellulose, hydrophilic polymers such as hydroxypropyl cellulose and the like. The nature of the “prolonged or controlled release material” may depend on whether the release properties are attained by a “prolonged release coating”. The term “prolonged release coating material” indicate that a material is used for obtaining a prolonged release coating.

The terms “prolonged release coating formulation” or “controlled release coating formulation” refer to a pharmaceutical composition including at least one prolonged release material or controlled release material, and at least one hydromorphone and naloxone or the pharmaceutically acceptable salts or derivatives thereof. The terms “prolonged release material” and “controlled release material” can be used interchangeably. In a “prolonged release coating formulation” or “controlled release coating formulation”, the “prolonged release material” or “controlled release material” are disposed on the pharmaceutically active agents to form a diffusion barrier. Typically, unlike in a matrix formulation, the actives are not intimately mixed with the prolonged release material and the prolonged release coating does not form a three dimensional structure within which the actives are distributed. As the term implies, the prolonged release material forms a layer above the actives. The pharmaceutically active agent is released from a prolonged release coating formulation over prolonged periods of time, such as, for example, 8, 10, 12, 14, 16, 18, 20, 22 or 24 hours.

It is to be understood that a material will be considered to act as prolonged or controlled release material if the dissolution profile of the pharmaceutically active agent(s) is slowed down compared to an immediate or conventional release formulation. If a prolonged or controlled release material can be used for manufacturing a prolonged or controlled release coating, it will be considered as a prolonged or controlled release coating material.

Pharmaceutically acceptable excipients which are used to adjust an already prolonged or controlled release to a specific profile are not necessarily considered to be prolonged or controlled release materials.

When it is mentioned that a prolonged release coating is disposed on pharmaceutically active agents, this is not to be construed as meaning that such a coating will necessarily be directly layered on such active pharmaceutically agents. Of course, if pharmaceutically active agents are layered on a carriers such as nu-pareil beads, the coating may be disposed directly thereon.

A pharmaceutical composition with a controlled or prolonged release coating may be obtained by combining the pharmaceutically active agents with carriers such as non-pareil beads and disposing a prolonged release coating on such combinations. Such coating may be made from polymers such cellulose ethers with ethyl cellulose being preferred, acrylic resins, other polymers and mixtures thereof. Such controlled or prolonged release coatings may comprise additional excipients such as pore-formers, binders and the like.

The present invention as disclosed herein with respect to all aspects and embodiments is meant to encompass the use of any pharmaceutically acceptable salt or derivative of hydromorphone and naloxone. Any embodiment of the invention referring to hydromorphone and naloxone is also meant to refer to salts and preferably the hydrochloride salts thereof unless indicated otherwise.

Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

Pharmaceutically acceptable derivatives of hydromorphone and naloxone include esters thereof as well as modified forms such as glycosylated, pegylated or hesylated forms of hydromorphone and naloxone.

If in the following reference is made to a pharmaceutically active agent such as hydromorphone, this always also includes the reference to a pharmaceutically acceptable salt or derivative of the free base of this pharmaceutically active agent unless it is specifically indicated that the reference to the pharmaceutically active agent, such as use of the term “hydromorphone” should only refer to the free base.

The use of the hydrochloride salts of both hydromorphone and naloxone is preferred.

In a preferred embodiment, the pharmaceutical dosage forms comprise hydromorphone or a pharmaceutically acceptable salt or derivative thereof or naloxone or a pharmaceutically acceptable salt or derivative thereof as the sole pharmaceutically active agents.

The pharmaceutical compositions may comprise about 1 to about 64 mg such as about 1 mg, about 2 mg, 3 mg, about 4 mg, about 8 mg, about 12 mg, about 16 mg, about 24 mg, about 32 mg, about 40 mg, about 48 mg or about 64 mg hydromorphone hydrochloride or equimolar amounts of any other pharmaceutically acceptable salt or derivative including but not limited to hydrates and solvates or of the free base. Where reference is made to amounts of hydromorphone hydrochloride this relates to anhydrous hydromorphone hydrochloride. If a hydrated version of hydromorphone hydrochloride is used, this will be used in an amount equivalent to the afore-mentioned amounts of anhydrous hydromorphone hydrochloride.

The pharmaceutical compositions may comprise about 0.5 to about 256 mg, such as about 0.5 mg, about 0.75 mg, about 1 mg, about 1.5 mg, about 2 mg, about 4 mg, about 8 mg, about 12 mg, about 16 mg, about 24 mg, about 32 mg, about 48 mg, about 64 mg, about 96 mg, about 128 or about 256 mg of naloxone hydrochloride or equimolar amounts of any other pharmaceutically acceptable salt, derivative or form including but not limited to hydrates and solvates or of the free base. Where reference is made to amounts of naloxone hydrochloride this relates to anhydrous naloxone hydrochloride. If a hydrated version of naloxone hydrochloride is used, this will be used in an amount equivalent to the afore-mentioned amounts of anhydrous naloxone hydrochloride.

Throughout this specification, the cited weight ratios of hydromorphone to naloxone refer to their respective hydrochloride salts—this has been done for illustrative purposes only. Regardless of chemical form of hydromorphone and naloxone used in a particular embodiment of the present invention, such as a salt, hydrate or base form of the molecule, the relative amounts of the molecule may also be expressed as a molar ratio. For example, a weight ratio of hydromorphone hydrochloride to naloxone hydrochloride of 4:1 may also be expressed as a molar ratio of 4.59:1. Similarly, a weight ratio of hydromorphone hydrochloride to naloxone hydrochloride of 2:1 may also be expressed as a molar ratio of 2.29:1.

In some embodiments, the present invention is directed to a prolonged release pharmaceutical coated bead composition comprising at least hydromorphone or a pharmaceutically acceptable salt or derivative thereof or naloxone or a pharmaceutically acceptable salt or derivative thereof and at least one prolonged release material which is preferably combined with these pharmaceutically active agents; wherein the amount of hydromorphone or a pharmaceutically acceptable salt or derivative thereof and/or naloxone or a pharmaceutically acceptable salt or derivative thereof released in vitro in 500 or 900 ml of Simulated Gastric Fluid, pH 1.2 using the Ph. Eur. paddle method at 100 rpm at 37° C. is:

at 1 h: 25 to 55% by weight of the pharmaceutically active agents,

at 2 h: 45 to 75% by weight of the pharmaceutically active agents,

at 3 h: 55 to 85% by weight of the pharmaceutically active agents,

at 4 h: 60 to 90% by weight of the pharmaceutically active agents,

at 6 h: 70 to 100% by weight of the pharmaceutically active agents,

at 8 h: more than 85% by weight of the pharmaceutically active agents,

at 10 h: more than 90% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio of equal to or less than about 4:1, or in the range of from about 3:1 to about 1:2, preferably in the range of from about 3:1 to about 1:2, such as a weight ratio of about 2:1, about 1:1 or about 1:2.

In some embodiments, the present invention is directed to a prolonged release pharmaceutical coated bead composition comprising at least hydromorphone or a pharmaceutically acceptable salt or derivative thereof or naloxone or a pharmaceutically acceptable salt or derivative thereof and at least one prolonged release material; wherein the amount of hydromorphone and/or a pharmaceutically acceptable salt or derivative thereof or naloxone or a pharmaceutically acceptable salt or derivative thereof released in vitro in 500 or 900 ml of Simulated Gastric Fluid, pH 1.2 using the Ph. Eur. paddle method at 100 rpm at 37° C. is:

at 1 h: 30 to 50% by weight of the pharmaceutically active agents,

at 2 h: 50 to 70% by weight of the pharmaceutically active agents,

at 3 h: 60 to 80% by weight of the pharmaceutically active agents,

at 4 h: 65 to 85% by weight of the pharmaceutically active agents,

at 6 h: 75 to 95% by weight of the pharmaceutically active agents,

at 8 h: more than 90% by weight of the pharmaceutically active agents,

at 10 h: more than 95% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio of equal to or less than about 4:1, or in the range of from about 3:1 to about 1:2, preferably in the range of from about 3:1 to about 1:2, such as a weight ratio of about 2:1, about 1:1 or about 1:2.

In some embodiments, the present invention is directed to a prolonged release pharmaceutical coated bead composition comprising at least hydromorphone or a pharmaceutically acceptable salt or derivative thereof or naloxone or a pharmaceutically acceptable salt or derivative thereof and at least one prolonged release material which is preferably combined with these pharmaceutically active agents; wherein the amount of hydromorphone or a pharmaceutically acceptable salt or derivative thereof and/or naloxone or a pharmaceutically acceptable salt or derivative thereof released in vitro in 500 or 900 ml of Simulated Gastric Fluid, pH 1.2 using the Ph. Eur. paddle method at 100 rpm at 37° C. is:

at 1 h: 10 to 30% by weight of the pharmaceutically active agents,

at 2 h: 34 to 54% by weight of the pharmaceutically active agents,

at 3 h: 53 to 73% by weight of the pharmaceutically active agents,

at 4 h: 65 to 85% by weight of the pharmaceutically active agents,

at 6 h: 75 to 95% by weight of the pharmaceutically active agents,

at 8 h: 80 to 100% by weight of the pharmaceutically active agents,

at 10 h: more than 90% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio of equal to or less than about 4:1, or in the range of from about 3:1 to about 1:2, preferably in the range of from about 3:1 to about 1:2, such as a weight ratio of about 2:1, about 1:1 or about 1:2.

In some embodiments, the present invention is directed to a prolonged release pharmaceutical coated bead composition comprising at least hydromorphone or a pharmaceutically acceptable salt or derivative thereof or naloxone or a pharmaceutically acceptable salt or derivative thereof and at least one prolonged release material which is preferably combined with these pharmaceutically active agents; wherein the amount of hydromorphone or a pharmaceutically acceptable salt or derivative thereof and/or naloxone or a pharmaceutically acceptable salt or derivative thereof released in vitro in 500 or 900 ml of Simulated Gastric Fluid, pH 1.2 using the Ph. Eur. paddle method at 100 rpm at 37° C. is:

at 1 h: 5 to 45% by weight of the pharmaceutically active agents,

at 2 h: 15 to 55% by weight of the pharmaceutically active agents,

at 3 h: 30 to 70% by weight of the pharmaceutically active agents,

at 4 h: 35 to 75% by weight of the pharmaceutically active agents,

at 6 h: 40 to 80% by weight of the pharmaceutically active agents,

at 8 h: 50 to 90% by weight of the pharmaceutically active agents,

at 10 h: 60 to 100% by weight of the pharmaceutically active agents,

at 12 h: 65 to 100% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio of equal to or less than about 4:1, or in the range of from about 3:1 to about 1:2, preferably in the range of from about 3:1 to about 1:2, such as a weight ratio of about 2:1, about 1:1 or about 1:2.

Preferably, the amount of the pharmaceutically active agents released in vitro in 500 or 900 ml of Simulated Gastric Fluid, pH 1.2 using the Ph. Eur. paddle method at 100 rpm at 37° C. is:

at 1 h: 8 to 42% by weight of the pharmaceutically active agents,

at 2 h: 18 to 52% by weight of the pharmaceutically active agents,

at 3 h: 33 to 67% by weight of the pharmaceutically active agents,

at 4 h: 38 to 72% by weight of the pharmaceutically active agents,

at 6 h: 43 to 77% by weight of the pharmaceutically active agents,

at 8 h: 53 to 87% by weight of the pharmaceutically active agents,

at 10 h: 63 to 97% by weight of the pharmaceutically active agents,

at 12 h: 73 to 100% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio of equal to or less than about 4:1, or in the range of from about 3:1 to about 1:2, preferably in the range of from about 3:1 to about 1:2, such as a weight ratio of about 2:1, about 1:1 or about 1:2.

More preferably, the amount of the pharmaceutically active agents released in vitro in 500 or 900 ml of Simulated Gastric Fluid, pH 1.2 using the Ph. Eur. paddle method at 100 rpm at 37° C. is:

at 1 h: 15 to 37% by weight of the pharmaceutically active agents,

at 2 h: 25 to 47% by weight of the pharmaceutically active agents,

at 3 h: 38 to 62% by weight of the pharmaceutically active agents,

at 4 h: 42 to 66% by weight of the pharmaceutically active agents,

at 6 h: 50 to 74% by weight of the pharmaceutically active agents,

at 8 h: 60 to 84% by weight of the pharmaceutically active agents,

at 10 h: 68 to 92% by weight of the pharmaceutically active agents,

at 12 h: 78 to 100% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio of equal to or less than about 4:1, or in the range of from about 3:1 to about 1:2, preferably in the range of from about 3:1 to about 1:2, such as a weight ratio of about 2:1, about 1:1 or about 1:2.

Even more preferably, the amount of the pharmaceutically active agents released in vitro in 500 or 900 ml of Simulated Gastric Fluid, pH 1.2 using the Ph. Eur. paddle method at 100 rpm at 37° C. is:

at 1 h: 19 to 33% by weight of the pharmaceutically active agents,

at 2 h: 29 to 43% by weight of the pharmaceutically active agents,

at 3 h: 43 to 47% by weight of the pharmaceutically active agents,

at 4 h: 47 to 61% by weight of the pharmaceutically active agents,

at 6 h: 55 to 69% by weight of the pharmaceutically active agents,

at 8 h: 65 to 79% by weight of the pharmaceutically active agents,

at 10 h: 73 to 87% by weight of the pharmaceutically active agents,

at 12 h: 83 to 100% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio of equal to or less than about 4:1, or in the range of from about 3:1 to about 1:2, preferably in the range of from about 3:1 to about 1:2, such as a weight ratio of about 2:1, about 1:1 or about 1:2.

Even more preferably, the amount of the pharmaceutically active agents released in vitro in 500 or 900 ml of Simulated Gastric Fluid, pH 1.2 using the Ph. Eur. paddle method at 100 rpm at 37° C. is:

at 1 h: 1 to 15% by weight of the pharmaceutically active agents,

at 2 h: 6 to 26% by weight of the pharmaceutically active agents,

at 3 h: 15 to 35% by weight of the pharmaceutically active agents,

at 4 h: 25 to 45% by weight of the pharmaceutically active agents,

at 6 h: 40 to 60% by weight of the pharmaceutically active agents,

at 8 h: 55 to 75% by weight of the pharmaceutically active agents,

at 10 h: 60 to 80% by weight of the pharmaceutically active agents,

at 12 h: 70 to 100% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio of equal to or less than about 4:1, or in the range of from about 3:1 to about 1:2, preferably in the range of from about 3:1 to about 1:2, such as a weight ratio of about 2:1, about 1:1 or about 1:2.

Storage under stressed conditions in the context of the present invention means that a pharmaceutical composition is subjected to increased temperature and/or relative humidity (RH) for prolonged periods of time. For example, typical stressed conditions refer to storage over at least one, two, three, four, five, six, twelve or eighteen months at 25° C. and 60% RH. Other stressed conditions refer to storage over at least one, two, three, four, five, six or twelve months at 30° C. and 65% RH Other stressed conditions refer to storage over at least one, two, three, four, five or six months at 40° C. and 75% RH.

Such stressed storage conditions are used to determine whether a pharmaceutical composition has a shelf life sufficient for long time storage under conditions as they are common in patients' households without negative effects on its safety and efficacy. Such negative effects may include that the in-vitro release rates change over time so that the efficacy of the composition is affected as different amounts of actives are released after administration. Similarly, negative effects may also result from degradation of the pharmaceutically active agents which may either decrease the overall amount of functional pharmaceutically active agent or lead to formation of toxic by-products.

If changes in the in vitro release profile or with respect to the amount of the active agent(s) of a pharmaceutical composition are observed after storage under stressed conditions, this may be indicative of stability problems. If such changes are not observed, this means vice versa that the pharmaceutical composition is storage stable.

The above mentioned stressed storage conditions can be used to estimate whether a pharmaceutical dosage will have a shelf life of at least about 12 months, at least about 18 months, at least about 24 months or at least about 36 months. Usually a shelf life of 18 months or more may be desirable as this is usually better compatible with e.g. supply of excipients, actives etc. for manufacturing purposes. If a pharmaceutical composition is storage stable, i.e. has essentially the same release rate after storage over at least one, two, three, four, five or more months at 25° C. and 60% RH, this will be usually indicative of shelf life of at least about 12 months. If a pharmaceutical composition is storage stable, i.e. has essentially the same release rate after storage over at least one, two, three, four, five or more months at 30° C. and 65% RH, this will be usually indicative of shelf life of at least about 18 months. If a pharmaceutical composition is storage stable, i.e. has essentially the same release rate after storage over at least one, two, three, four, five or more months at 40° C. and 75% RH, this will be usually indicative of a shelf life of at least about 24 months such as 36 months.

The term “substantially the same release rate” refers to the situation where the in vitro release rate for a pharmaceutical composition which has been subjected to stressed conditions is compared to a reference composition. The reference composition is an identical pharmaceutical composition which, however, has not been subjected to stressed conditions. If the in vitro release profile of the composition subjected to stressed conditions does not deviate by more than about 20%, preferably by no more than about 15%, more preferably by no more than 10% and even more preferably by no more than about 5% from the in vitro release profile of the reference composition, the in-vitro release rate is considered to be substantially the same.

The term “hydromorphone and/or naloxone related substances” or the like refers to substances that arise from chemical reactions of hydromorphone or naloxone, pharmaceutically acceptable salts and derivatives thereof such as e.g. degradation. These substances can be distinguished as known hydromorphone related substances where the identity of the substance and its origin is known, as known naloxone related substances where the identity of the substance and its origin is known, and as unknown substances. For unknown substances, their identity is not known. However, it is assumed that they arise from hydromorphone and/or naloxone, pharmaceutically acceptable salts and derivatives thereof. It is to be understood that the term “hydromorphone and naloxone related substances” includes the sum of known hydromorphone related substances, known naloxone related substances and unknown substances and is thus equivalent to the term “total hydromorphone and naloxone related substances”.

Terms like “less than about 4% of substances related to hydromorphone and naloxone, or to pharmaceutically acceptable salts or derivatives thereof” or “less than about 3% of substances related to hydromorphone and naloxone or to pharmaceutically acceptable salts or derivatives thereof” etc. indicate that the amount of total substances as described in the preceding paragraph is less than e.g. 4% or 3% by weight based on the total amount of the active ingredient which is present in lower amounts (i.e. hydromorphone or naloxone), or a pharmaceutically acceptable salt or derivative thereof which is present in the pharmaceutical composition in the lower amount. Thus, if a pharmaceutical composition comprises hydromorphone HCl and naloxone HCl in 1:2 ratio by weight, the amount of total substances is calculated from the sum of known hydromorphone HCl related substances, known naloxone HCl related substances and unknown substances which is then referenced to the amount of hydromorphone HCl. If a pharmaceutical composition comprises hydromorphone HCl and naloxone HCl in 2:1 ratio by weight, the amount of total substances is calculated from the sum of known hydromorphone HCl related substances, known naloxone HCl related substances and unknown substances which is then referenced to the amount of naloxone HCl.

“Known hydromorphone related substances” include hydromorphone n-oxide, noroxymorphone, pseudohydromorphone.

“Known naloxone related substances” include noroxymorphon, 10a-hydroxynaloxon, 7,8-didehydronaloxon, pseudonaloxon, 3-o-allylnaloxon.

Terms like “less than 4% of known substances related to hydromorphone, or to pharmaceutically acceptable salts or derivatives thereof” or “less than 3% of known substances related to hydromorphone, or to pharmaceutically acceptable salts or derivatives thereof” etc. indicate that the amount of known hydromorphone related substances is less than e.g. 4% or 3% of known hydromorphone related substance by weight based on the total amount of hydromorphone, or a pharmaceutically acceptable salt or derivative thereof in the composition.

Terms like “less than 4% of known substances related to naloxone, or to pharmaceutically acceptable salts or derivatives thereof′ or “less than 3% of known substances related to naloxone, or to pharmaceutically acceptable salts or derivatives thereof′ etc. indicate that the amount of known naloxone related substances is less than e.g. 4% or 3.0% of known naloxone related substance by weight based on the total amount of naloxone, or a pharmaceutically acceptable salt or derivative thereof in the composition.

In order to assess stability one may subject a pharmaceutical composition to stressed conditions as mentioned above and determine the amount of total hydromorphone and/or naloxone related substances. One then determines the amount of total hydromorphone and/or naloxone related substances for an identical pharmaceutical composition which has not been subjected to stressed conditions. This composition is considered to be a reference composition. The detection of “total hydromorphone related and/or naloxone substances” is typically performed by HPLC analysis using e.g. CAT columns. The amount of the substances including the amount of unknown substances is then determined by calculating the area under the respective peaks in the chromatogram. The identity of substances can be determined by doing the same analysis with pure known reference substances. In a further aspect the present invention aims at providing pharmaceutical compositions which after storage under stressed conditions have less than 4%, less than 3%, less than 2%, less than 1%, less than 0.5%, less than 0.2% or even less than 0.1% of total substances related to hydromorphone or a pharmaceutically acceptable salt or derivative thereof and/or related to naloxone or a pharmaceutically acceptable salt or derivative thereof

In a further aspect the present invention aims at providing pharmaceutical compositions which after storage under stressed conditions have less than 1% such as less than 0.5%, less than 0.4%, less than 0.3%, less than 0.2%, less than 0.1% or even less than 0.05% of known substances related to hydromorphone or a pharmaceutically acceptable salt or derivative thereof and less than 1% such as less than 0.5% of known substances related to naloxone or a pharmaceutically acceptable salt or derivative thereof.

Stressed storage conditions may be the same as mentioned above. Thus typical stressed conditions may refer to storage over at least one, two, three, four, five or six months at 25° C. and 60% RH, at 30° C. and 65% RH or at 40° C. and 75% RH.

A pharmaceutical composition will thus be considered to be stable if after subjecting it to stressed conditions, it has no more than about 4% such as no more than about 3%, preferably no more than about 2%, more preferably no more than about 1% and even more preferably no more than about 0.5% of hydromorphone and/or naloxone related substances.

The prolonged release compositions in accordance with the invention may be formulated into different dosage forms. For example, prolonged release compositions may take the form of tablets or mini-tablets. Tablets may be a monolithic tablet comprising, for example, a continuous prolonged release matrix. However, tablets or mini-tablets may be also be made from multiparticulates which are compressed into tablets. Such multiparticulates may, for example, comprise a prolonged release matrix optionally with an immediate release phase or active loaded beads with a prolonged release coating and optionally an immediate release phase thereon. The dosage form may also take the form of such multiparticulates, for example, granules or mini-tablets which may be filled into a capsule.

The in vitro release rates of the prolonged release pharmaceutical compositions will be chosen such that a therapeutic efficacy in vivo is achieved over preferably at least twelve hours and in some instance even up to twenty four hours. Such compositions may be described as “twice a day” or “once a day” formulations as they may be administered on such a regimen.

The prolonged release material may be any material that is known to be capable of imparting controlled release properties on the active agent.

Such materials may be hydrophilic and/or hydrophobic materials such as gums, cellulose ethers, acrylic polymers, protein-derived materials and the like.

Prolonged materials may also include fatty acids, fatty alcohols, glyceryl esters of fatty acids, polyethylene glycols, mineral and oils and waxes. Fatty acids and fatty alcohols preferable are those with a C₁₀ to C₃₀ chain, preferably with a C₁₂ to C₂₄ chain and more preferably with a C₁₄ to C₂₀ chain or a C₁₆ to C₂₀ chain. Materials such as stearyl alcohol, cetostearyl alcohol, cetyl alcohol, myristyl alcohol and polyalkylene glycols may be preferred. Waxes may be selected from natural and synthetic waxes such as beeswax, carnauba wax. Oils may be vegetable oils and include, for example, castor oil.

The prolonged release matrix materials which may be considered in the context of the present invention may also be selected from cellulose ethers.

The term “cellulose ethers” comprises cellulose-derived polymers derivatized with at least alkyl and/or hydroxyalkyl groups which may be hydrophilic or hydrophobic.

For example, the prolonged release matrix material may be a hydrophilic hydroxy alkyl cellulose such as a hydroxy (C₁-C₆) alkyl celluloses such as hydroxypropyl cellulose, hydroxypropylmethyl cellulose and particularly preferably hydroxyethyl cellulose.

Examples of hydrophobic cellulose ethers include e.g. ethyl cellulose. The use of ethyl cellulose may be preferred. Hydrophobic cellulose ethers such as ethyl cellulose may be particularly suitable for imparting alcohol resistance to pharmaceutical compositions.

A particularly suitable material for prolonged release matrix formulations in accordance with the present invention may be selected from the group of acrylic resins. Such acrylic resins may be made from (meth)acrylic acid (co) polymers.

There are various types of (meth)acrylic acid (co)polymers available which may be characterised according to the nature of their residues such as neutral (meth)acrylic acid (co)polymers, (meth)acrylic acid (co)polymers with anionic residues or (meth)acrylic acid ester copolymers with cationic residues.

Neutral (meth)acrylic acid (co)polymers include polymers having 95 to 100% by weight of polymerised monomers having neutral residues. Monomers with neutral residues can be C₁-C₄ alkyl esters of acrylic or methacrylic acid such as methylmethacrylate, ethylmethacrylate, butylmethacrylate, methylacrylate, ethylacrylate and butylacrylate. For example, neutral (meth)acrylic acid (co)polymers may comprise 20 to 40% by weight ethylacrylate and 60 to 80% by weight methylmethacrylate. Such polymers are, for example, available under the trade name Eudragit® NE which is a copolymer of 30% by weight ethylacrylate and 70% by weight methylmethacrylate. This polymer is usually provided in the form of a 30% or 40% aqueous dispersion (Eudragit® NE 30 D, Eudragit® NE 40 D or Eudragit® NM 30 D).

(Meth)acrylic acid (co)polymers with functional anionic residues may be (meth)acrylic acid (co)polymers having 25 to 95% by weight of radically polymerised C₁ to C₄ alkyl esters of acrylic or methacrylic acid and 5 to 75% by weight of methacrylate monomers with an anionic group in the alkyl residue. C₁ to C₄ alkyl esters of acrylic or methacrylic acid are again methylmethacrylate, ethyl methacrylate, butylmethacrylate, methylacrylate, ethylacrylate and butylacrylate. A (meth)acrylate monomer with an anionic group in the alkyl residue may be for example acrylic acid and preferably methacrylic acid. Such methacrylic acid copolymers with an anionic functional group may comprise e.g. 40 to 60% by weight methacrylic acid and 60 to 40% by weight methylmethacrylate or 60 to 40% by weight ethyl acrylate. These types of polymers are available as Eudragit® L100/Eudragit® L 12.5 or Eudragit® L 100-55/Eudragit® L 30 D-55, respectively.

For example, Eudragit® L 100 is a copolymer of 50% by weight methylmethacrylate and 50% by weight methacrylic acid. It is also provided as a 12.5% solution (Eudragit® L 12.5). Eudragit® L 100-55 is a copolymer of 50% by weight ethylacrylate and 50% by weight methacrylic acid. It is also provided as 30% dispersion (Eudragit® L 30 D-55).

(Meth)acrylic acid (co)polymers with an anionic functional group may also comprise 20 to 40% by weight methacrylic acid and 80 to 60% by weight methylmethacrylate. These types of polymers are usually available under the trade name Eudragit® S. It is also provided as a 12.5% solution (Eudragit® S 12.5). Another type of methacrylic acid copolymers with an anionic functional group is available under the trade name Eudragit® FS which typically comprises 10 to 30% by weight methylmethacrylate, 50 to 70% by weight methylacrylate and 5 to 15% by weight methacrylic acid. Thus, Eudragit®FS may be a polymer of 25% by weight methylmethacrylate, 65% by weight methylacrylate and 10% by weight methacrylic acid. It is usually provided as 30% dispersion (Eudragit® FS 30 D).

(Meth)acrylic acid (co)polymers with functional cationic groups may be methacrylic acid copolymers with tertiary amino groups. Such polymers may comprise 30% to 80% by weight of radically polymerised C₁-C₄ alkyl esters of acrylic acid or methacrylic acid and 70 to 20% by weight methacrylate monomers with a tertiary amino group in the alkyl rest.

Suitable monomers with a functional tertiary amino group are disclosed, for example, in U.S. Pat. No. 4,705,695 (see column 3, line 64 to column 4, line 13). They include for example dimethylaminoethyl acrylate, 2-dimethylaminopropyl acrylate, dimethylaminopropyl methacrylate, dimethylaminobenzyl acrylate, dimethylaminobenzyl methacrylate, (3-dimethylamino-2,2-dimethyl)propyl acrylate, dimethylamino-2,2-dimethylpropylmethacrylate, (3-diethylamino-2,2-dimethyl)propyl acrylate and diethylamino-2,2-dimethylpropylmethacrylate. Particularly suitable is dimethylaminoethyl methacrylate. The amount of monomers with a tertiary amino group in the copolymer may vary between 20 to 70%, between 40 to 60%. The amount of C₁ to C₄ alkyl esters of acrylic or methacrylic acid may be within 70 to 30% by weight. C₁ to C₄ alcohol esters of acrylic or methacrylic acid include methylmethacrylate, ethylmethacrylate, butylmethacrylate, methylacrylate, ethylacrylate and butylacrylate. A common (meth)acrylic acid (co)polymer with a tertiary amino group may comprise 20 to 30% by weight methylmethacrylate, 20 to 30% by weight butylmethacrylate and 60 to 40% by weight dimethylaminoethyl methacrylate. For example the commercially available Eudragit® E 100 comprises 25% by weight methylmethacrylate, 25% by weight butylmethacrylate and 50% by weight dimethylaminoethyl methacrylate. Another common commercially available polymer, Eudragit®E PO comprises copolymers of methylmethacrylate, butylmethacrylate and dimethylaminoethyl methacrylate in a ratio of 25:25:50.

Another type of (meth)acrylic acid (co)polymers with functional cationic groups is (meth)acrylic acid (co)polymers with a quaternary amino group. This type of (meth)acrylic acid (co)polymers typically comprises 50 to 70% of radically polymerised methylmethacrylate, 20 to 40% by weight of ethylacrylate and 12 to 2% by weight of 2-trimethylammoniumethyl methacrylate chloride. Such polymers are, for example, available under the trade names Eudragit®RS or Eudragit®RL.

For example, Eudragit®RS comprises radically polymerised units of 65% by weight methylmethacrylate, 30% by weight ethylacrylate and 5% by weight 2-trimethylamoniumethyl methacrylate chloride. Eudragit®RL comprises radically polymerised units of 60% by weight methylmethacrylate, 30% by weight ethylacrylate and 10% by weight 2-trimethylamoniumethyl methacrylate chloride.

The amount of prolonged release material(s) in the prolonged release formulation may be of about 5 to 90% by weight, of about 10 to 70% by weight, of about 20 to 60% by weight, of about 20% to about 55% by weight, of about 25% to about 50% by weight, of about 25% to about 45% by weight and preferably of about 30 to about 40% by weight based on the weight of the pharmaceutical composition. The amount of prolonged release material that is incorporated into the composition can be one way of adjusting the prolonged release properties. For example, if the amount of prolonged release material is increased, the release can be further prolonged. The aforementioned amounts refer to the overall content of prolonged release materials in a pharmaceutical composition. These amounts may thus refer to a mixture of various prolonged release materials such as a neutral (meth)acrylic acid (co)polymer, a hydrophobic cellulose ether and/or a fatty alcohol.

If cellulose ether is among the prolonged release materials, it will typically be present in an amount of about 5% to about 50% by weight, of about 5% to about 45% by weight, of about 5% to about 40% by weight, of about 5% to about 35% by weight, of about 5% to about 30% by weight, of about 5% to about 25% by weight, of about 5% to about 20% by weight such as of about 5% by weight, of about 7% by weight, of about 10% by weight, of about 15% by weight, of about 18% by weight or of about 20% by weight based on the weight of the pharmaceutical composition.

If fatty alcohol is among the prolonged release materials, it will typically be present in an amount of about 5% to about 50% by weight, of about 5% to about 45% by weight, of about 5% to about 40% by weight, of about 5% to about 35% by weight, of about 10% to about 30% by weight, of about 10% to about 25% by weight such as of about 10% by weight, of about 15% by weight, of about 20% by weight or about 25% by weight based on the weight of the pharmaceutical composition.

If (meth)acrylic acid (co)polymer is among the prolonged release materials, it will typically be present in an amount of about 5% to about 50% by weight, of about 5% to about 45% by weight, of about 5% to about 40% by weight, of about 5% to about 35% by weight, of about 10% to about 30% by weight, of about 10% to about 25% by weight such as of about 10% by weight, of about 15% by weight, of about 20% by weight or about 25% by weight based on the weight of the pharmaceutical composition.

The pharmaceutical compositions in accordance with the invention may also include pharmaceutically acceptable excipients such fillers, lubricants, binders, release rate modifiers, anti-tacking agents etc.

Fillers which may also be designated as diluents may include e.g. lactose, preferably anhydrous lactose, glucose or saccharose, starches, their hydrolysates, microcrystalline cellulose, cellatose, sugar alcohols such as sorbitol or mannitol, polysoluble calcium salts like calcium hydrogen phosphate, dicalcium- or tricalcium phosphate and combinations of two or more of the above fillers.

It has been observed that the combination of hydromorphone and naloxone can be moisture sensitive in particular if cellulose ethers are used as prolonged release material. In view of this situation it can be preferred to use fillers which do not import moisture e.g. in the form of water. In preferred embodiments one may thus use anhydrous fillers such as anhydrous lactose.

Lubricants can include highly dispersed silica, talcum, corn starch, magnesium oxide and magnesium- or calcium stearate, fats like hydrated castor oil, sodium stearyl fumarate and combinations of two or more of the above lubricants.

It can be preferred to use a combination of magnesium stearate and talcum as lubricants. It has been found that if appropriate amounts of these lubricants are chosen, one can e.g. improve flow properties of granules used for compressing.

It thus can be preferred to use a lubricant amount of about 0.5% to about 4% by weight, of about 0.7% to about 3% by weight, of about 1% to about 2% by weight such as of about 1.0% by weight, of about 1.1% by weight, of about 1.2% by weight, of about 1.3% by weight, of about 1.4% by weight, of about 1.5% by weight, of about 1.6% by weight, of about 1.7% by weight, of about 1.8% by weight, of about 1.9% by weight or of about 2.0% by weight based on the weight of the pharmaceutical composition. An amount of about 0.75% to about 1.25% by weight based on the weight of the pharmaceutical composition can be preferred, particularly if magnesium stearate and talc are used. The aforementioned amounts refer to the amount of all lubricants (i.e., including mixtures) in the composition.

Binders can include hydroxypropyl cellulose (HPC), hydroxypropyl methyl cellulose, polyvinyl pyrollidone, carbopol, and combinations thereof

It can be preferred to use HPC as a binder as this may positively influence the hardness of the tablets.

It thus can be preferred to use a binder amount of about 1% to about 10% by weight, of about 2% to about 9% by weight, of about 3% to about 7% by weight, of about 3% to about 6% by weight, of about 4% to about 5% by weight such as of about 4.0% by weight, of about 4.1% by weight, of about 4.2% by weight, of about 4.3% by weight, of about 4.4% by weight, of about 4.5% by weight, of about 4.6% by weight, of about 4.7% by weight, of about 4.8% by weight, of about 4.9% by weight or of about 5.0% by weight based on the weight of the pharmaceutical composition. An amount of about 4.4% to about 5.0% by weight based on the weight of the pharmaceutical composition can be preferred, particularly of HPC is used as binder. The aforementioned amounts refer to the amount of all binders (i.e. including mixtures) in the composition.

It can be preferred to not use povidone as a binder.

Release rate modifiers are pharmaceutically acceptable excipients which may be used to tune the release which otherwise would be obtained using the prolonged release materials, e.g. to accelerate the release or to further slow it down. Such release modifiers may be hydrophilic substances such as polyethylenglycols, hydroxypropylmethylcellulose, hydroxyethylcellulose, and the like or hydrophobic substances such as oils, waxes and the like. Other release modifiers may include some the aforementioned (meth)acrylic acid(co)polymers such as polymers of the Eudragit® RLPO type or gums such as xanthan gum.

Release rate modifiers such as polymers of the Eudragit/®RLPO type, low molecular weight hydroxypropylmethlycellulose such Hypromellose K100M or xanthan gum may be preferred.

Such release rate modifiers may be present in an amount of about 1% to about 20% by weight, of about 2% to about 19% by weight, of about 3% to about 18% by weight, of about 4% to about 17% by weight, of about 5% to about 15% by weight such as of about 5% by weight, of about 6% by weight, of about 7% by weight, of about 8% by weight, of about 9% by weight, of about 10% by weight, of about 11% by weight, of about 12% by weight, of about 13% by weight, of about 14% by weight or of about 15% by weight based on the weight of the pharmaceutical composition. The aforementioned amounts refer to the amount of all release rate modifiers (i.e. including mixtures) in the composition.

It is to be understood that the functions of pharmaceutically acceptable excipients may be overlapping. For example, a spheronising agent such as microcrystalline cellulose can also be used as filler if appropriate amounts are chosen. Further, HPMC may not only act as release rate modifying agent but also as binder if e.g. used in prolonged release formulation with a coating.

Prolonged release coatings may be made from materials which are common in the art.

They may thus be selected from e.g. prolonged release materials selected e.g. from (i) an alkylcellulose; (ii) an acrylic polymer; (iii) polyvinylalcohol or (iv) mixtures thereof. Hydrophobic representatives of the afore-mentioned groups can be preferred. The coating may be applied in the form of an organic or aqueous solution or dispersion.

In some embodiments, the controlled release coating is derived from an aqueous dispersion of the hydrophobic controlled release material. The coated composition can then be cured.

In preferred embodiments, the controlled release coatings include a plasticizer such as those described herein below.

In certain embodiments, one may coat with an amount of coating material which is sufficient to obtain a weight gain level from about 2 to about 20%, e.g., about 2 to about 15% and preferably about 5 to about 10% such as 6%, 7%, 8% or 9% in order to obtain sufficiently prolong the release from the formulation.

Cellulosic materials and polymers, including alkyl celluloses are prolonged release materials well suited for coating substrates, e.g., beads, granules, tablets, etc. according to the invention. Simply by way of example, one preferred alkyl cellulosic polymer is ethyl cellulose.

One commercially available aqueous dispersion of ethyl cellulose is Aquacoat® such as Aquacoat® ECD30 (FMC Corp., Philadelphia, Pa., U.S.A.). Aquacoat is prepared by dissolving the ethyl cellulose in a water-immiscible organic solvent and then emulsifying the same in water in the presence of a surfactant and a stabilizer. After homogenization to generate submicron droplets, the organic solvent is evaporated under vacuum to form a pseudo latex.

Another aqueous dispersion of ethyl cellulose is commercially available as Surelease® (Colorcon, Inc., West Point, Pa., U.S.A.). This product is prepared by incorporating plasticizer into the dispersion during the manufacturing process. A hot melt of a polymer, plasticizer (dibutyl sebacate or medium chain triglycerides), and stabilizer (oleic acid) is prepared as a homogeneous mixture, which is then diluted with an alkaline solution to obtain an aqueous dispersion which can be applied directly onto substrates.

In other of the present invention, the prolonged release coating material is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride) and glycidyl methacrylate copolymers.

In certain preferred embodiments, the acrylic polymer is comprised of one or more ammonium methacrylate copolymers. Ammonium methacrylate copolymers are well known in the art, and are described as fully polymerized copolymers of acrylic and methacrylic acid esters with a low content of quaternary ammonium groups. Typical examples include Eudragit® RS30D which is a low permeability ammonium methacrylate polymer and Eudragit®RL30D which is a high permeability ammonium methacrylate polymer. Eudragit RL and Eudragit RS are water swellable, and the amount of water absorbed by these polymers is pH-dependent, however, dosage forms coated with Eudragit RL and RS are pH-independent.

The acrylic coatings may comprise a mixture of two acrylic resin lacquers commercially available from Rohm Pharma under the Trade names Eudragit®RL30D and Eudragit® RS30D, respectively. The Eudragit®RL/RS dispersions of the present invention may be mixed together in any desired ration in order to ultimately obtain a prolonged-release formulation having a desirable dissolution profile.

Other polymers which can be used as a prolonged release coating materials if they are applied at sufficient amounts are, for example, hydrophilic polymers such as hydroxypropylmethylcellulose.

The above mentioned coatings may also be applied in combination. Further it is possible to influence the release properties of a dosage form by increasing the amount of the coating material and thus the thickness of the coating.

In embodiments of the present invention where the coating comprises an aqueous dispersion of a hydrophobic controlled release material, the inclusion of an effective amount of a plasticizer in the aqueous dispersion of hydrophobic material may further improve the physical properties of the prolonged release coating. For example, because ethyl cellulose has a relatively high glass transition temperature and may not form flexible films under normal coating conditions, it can be preferred to incorporate a plasticizer into an ethyl cellulose coating containing prolonged release coating before using the same as a coating material. Generally, the amount of plasticizer included in a coating solution is based on the concentration of the film-former, e.g., most often from about 1 to about 50% by weight of the film-former.

Examples of suitable plasticizers for ethyl cellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit®RL/RS lacquer solutions include polyethylene glycols, propylene glycol, diethyl phthalate, castor oil and triacetin.

The pharmaceutical compositions in accordance with the invention as described herein may be formulated to provide a mean AUCt of about 1162 h*pg/ml to about 2241 h*pg/ml and preferably of about 1328 to about 2075 h*pg/ml per mg administered amount of hydromorphone and a mean Cmax of about 122 pg/ml to about 234 pg/ml and preferably of about 139 to about 218 pg/ml per mg administered amount of hydromorphone and mean tmax of about 1 h to about 4.5 h, preferably of about 1.5 h to about 4 h and more preferably of about 1.5 h to about 3 h. These values refer preferably to single dose administration to healthy subjects. Preferably, administration is in the fasted state. The mean values of Cmax, AUCt and tmax refer to the geometric mean.

The pharmaceutical compositions in accordance with the invention as described herein (particularly the coated bead embodiment) may be formulated to provide a mean AUCt of about 5.900 ng*h/mL to about 8.400 ng*h/mL and preferably of about 6.500 to about 8.400 ng*hg/mL per mg administered amount of hydromorphone and a mean Cmax of about 0.390 ng/ml to about 0.726 ng/mL and preferably of about 0.590 to about 0.726 ng/mL per mg administered amount of hydromorphone and mean tmax of about 1 h to about 4.5 h, preferably of about 1.5 h to about 4 h and more preferably of about 4.0 h to about 6.5 h. These values refer preferably to single dose administration to healthy subjects. Preferably, administration is in the fasted state. The mean values of Cmax, AUCt and tmax refer to the geometric mean.

The “Cmax value” indicates the maximum blood plasma concentration of the active agent hydromorphone.

The “tmax value” indicates the time point at which the Cmax value is reached. In other words, tmax is the time point of the maximum observed plasma concentration.

The “AUC (Area Under the Curve)” value corresponds to the area of the concentration curve. The AUC value is proportional to the amount of the active agent absorbed into the blood circulation in total and is hence a measure for the bioavailability.

The “AUCt value” is the value for the area under the plasma concentration-time curve from the time of administration to the last measurable concentration. AUCt values are usually calculated using the linear trapezoidal method.

If pharmacokinetic parameters such as mean tₘₐₓ, cₘₐₓ and AUCt are measured for healthy subjects which may be healthy human, they are typically obtained by measuring the development of blood plasma values over time in a test population of approximately 16 to 24 healthy human subjects. Regulatory bodies such as the European Agency for the Evaluation of Medicinal Products (EMEA) or the Food and Drug Administration (FDA) will usually accept data obtained from e.g. 16 or 24 test persons. However, initial trials involving fewer participants such as 8 to 16 participants may also be acceptable.

The term “healthy” subjects in this context refers to a typical male or female of usually Caucasian origin with average values as regards height, weight and physiological parameters such as blood pressure etc. Healthy human subjects for the purposes of the present invention are selected according to inclusion and exclusion criteria which are based on and in accordance with recommendations of the International Conference on Harmonization of Clinical Trials (ICH).

For the purposes of the present invention, healthy subjects may be identified according to conventional inclusion and exclusion criteria.

Thus, inclusion criteria comprise, for example, an age between ≧18 and ≦45 years; a BMI within the range 19-29 kg/m², and within the weight range 60-100 kg for males and 55-90 kg for females; that females must be non-nursing, non-pregnant, and provide a negative urine β-hCG pregnancy test within 24 hours before receiving the study medication; generally good health, evidenced by a lack of significantly abnormal findings on medical history, physical examination, clinical laboratory tests, vital signs, and ECG and the like.

Exclusion criteria comprise, for example, exposure to any investigational drug or placebo within 3 months of the first dose of study medication, any significant illness within the 30 days before the first dose of study medication, any clinically significant abnormalities identified at prestudy screening for medical history, physical examination or laboratory analyses, use of any prescription medication (except HRT for postmenopausal females and contraceptive medication) in the 21 days, or over the counter medication including acid controllers, vitamins, herbal products and/or mineral supplements in the 7 days, before first dose of study medication, concurrent medical condition known to interfere with gastrointestinal drug absorption (e.g. delayed gastric emptying, mal absorption syndromes), distribution (e.g. obesity), metabolism or excretion (e.g. hepatitis, glomerulonephritis), history of or concurrent medical condition, which in the opinion of the investigator would compromise the ability of the subject to safely complete the study, history of seizure disorders for which subjects required pharmacologic treatment, current history of smoking more than 5 cigarettes a day, subjects with evidence of active or past history of substance or alcohol abuse according to DSM-IV criteria, subjects who reported regular consumption of 2 or more alcoholic drinks per day or have blood alcohol levels of ≧0.5% at screening, donation of more than 500 mL of blood or blood products or other major blood loss in the 3 months before first dose of study medication, any positive results in the prestudy screen for ethanol, opiates, barbiturates, amphetamines, cocaine metabolites, methadone, propoxyphene, phencyclidine, benzodiazepines, and cannabinoids in the specimen of urine collected at screening, known sensitivity to hydromorphone, naloxone, or related compounds and the like.

The pharmaceutically acceptable excipients may include the fillers, binders, lubricants, release rate modifiers, spheronising agents, anti-tacking agents, etc. as mentioned above. However, some of these excipients such as, for example, lubricants may be added at a later stage.

Different technology is available to obtain such granules. One may use, for example, drum granulation or fluidized bed granulation.

The granules which may be produced by wet granulation extrusion may be dried before being mixed with the at least one pharmaceutically active agent.

Typically, drying takes place at humidity in the range of about 0.5% to about 5.0% at a temperature in the range of about 20° C. to about 90° C. and for a time in the range of about 10 min to about 3 hours. Drying at ambient humidity at a temperature in the range of about 40° C. to about 90° C. and for a time in the range of about 15 min to about 2 hours can be preferred.

The granules may then be optionally screened in order to select granules of substantially uniform size. Selecting granules of substantially uniform size before compressing them may improve the prolonged release properties of the final prolonged release pharmaceutical composition as the active and the granules are then assumed to be more uniformly distributed which may prevent irregularities in the release profile. Granules for which at least about 70%, preferably at least about 80%, more preferably at least about 90% are of about the same mean size will typically be considered as being of substantially uniform size.

Preferably, granules are selected of a mean size in the range of about 100 μm to about 2 mm, more preferably in the range of about 100 μm to about 1 mm, and even more preferably in the range of about 100 μm to about 600 μm. Selection may be performed using a sieve with an appropriate mesh size.

In some embodiments the granules may be milled before selecting them for their size. Milling may both increase the yield of the selection step and improve the granules' suitability for the subsequent compression step. For milling one may use for example a rotary hammer mill or top/bottom driven conical mill.

For compressing the pharmaceutically active agent(s) with the granules, one may use typical tabletting equipment such as Example Fette or Kilian press.

When compressing granules and active(s), one may also include pharmaceutically acceptable excipients as they are commonly used in the art. For example, one may add lubricants, anti-tacking agents, binders and the like. For lubricants, the use of magnesium stearate and/or talc in the aforementioned amounts can be of advantage.

As mentioned above, prolonged release pharmaceutical dosage forms in accordance with the invention may be additionally subjected to a heat treatment step as has been described above.

The prolonged release coating may be produced by methods common in the art such a fluidized bed spraying.

Various embodiments of the present application will be illustrated with reference to the following non-limiting examples which should not be used to construe the scope of the invention.

### Example 1

In this example, there is described a randomized, double-blind, placebo-controlled, dose-ranging crossover study evaluating the effect of naloxone on intravenous hydromorphone abuse potential in healthy, non-dependent, opioid-experienced recreational drug users.

### Overall Design

This was a single-centre, double-blind, randomized crossover dose-ranging study to assess the appropriate naloxone to HMO (HMO) ratio required to block the pharmacodynamic (PD) effects of a fixed intravenous dose of hydromorphone. The study consisted of a standard medical screening visit, a double-blind qualification phase, which included a naloxone challenge to determine physical dependence, a treatment phase and a safety follow-up visit.

### Participants

Eligible subjects were healthy male and female adult volunteers, between 18 and 55 years of age, inclusive, with a body mass index (BMI) between 18.0 and 29.9 kg/m², inclusive. Subjects were non-dependent recreational drug users with moderate opioid experience, defined as having used opioids for non-therapeutic purposes on at least 10 occasions in the past year and had used opioids at least 3 times in the 12 weeks prior to the medical screening visit.

### Qualification Phase

Upon admission to the qualification phase, a naloxone challenge test (Saddock et al., 2005) using the Objective Opioid Withdrawal Scale (Handelsman et al., 1987) was administered to confirm that subjects were not opioid-dependent, and to ensure that subjects would not undergo withdrawal during the treatment phase, when HMO was co-administered with naloxone.

The purpose of this double-blind, placebo-controlled two-way crossover qualification phase was to enrich the subject sample with self-reported opioid users who could safely tolerate the selected dose of HMO (30 μg/kg), demonstrate an appropriate response to placebo, and discriminate the effects of a single intravenous dose of HMO compared to placebo in a laboratory setting. Specifically, subjects had to achieve a peak response (Eₘₐₓ) to HMO that was at least 20% greater than that of placebo on “at this moment” Drug Liking visual analog scale (VAS) and Addiction Research Center Inventory Morphine-Benzedrine Group (ARCI-MBG) scale.

### Treatment Phase

The treatment phase consisted of six separate visits, during which subjects received a single intravenous dose of naloxone (placebo, 1.875 μg/kg, 3.75 μg/kg, 5 μg/kg, 7.5 μg/kg, or 15 μg/kg) followed by a fixed dose of HMO (30 μg/kg, intravenous). The resulting HMO to active naloxone ratios were: 16:1, 8:1, 6:1, 4:1 and 2:1, respectively. The order of naloxone doses was randomized across subjects according to a 6×6 Latin square. Pharmacodynamic, pharmacokinetic and safety assessments were conducted up to 8 hours post-infusion.

### Pharmacodynamic Assessments

‘At this moment’ subjective measures VAS of Drug Liking (bipolar), High, Good Drug Effects, Bad Drug Effects, Feeling Sick, Drowsiness/Alertness (bipolar), and Any Drug Effects, and ARCI-MBG were administered at pre-infusion and at 5, 15, 30 and 45 minutes post-infusion and 1, 1.5, 2, 3, 4, 5 and 8 hours post-infusion. Scales assessing drug effects were not administered pre-infusion. Overall Drug Liking VAS, Take Drug Again VAS and Subjective Drug Value (SDV) are global measures that were administered only at 8 hours post-infusion. Each unipolar VAS was scored as an integer from 0 to 100, with anchors such as “Definitely not” (score=0) to “Definitely so” (score=100). Bipolar scales of Drug Liking used anchors “Strong Disliking” (score=0) and “Strong Liking” (score=100), with a score of 50 being neutral. The ARCI-MBG 16-item subscale was used to measure positive subjective effects (Martin 1971). The SDV is a procedure to determine the theoretical monetary value of the dose of study drug administered, adapted from Griffiths et al. (1993, 1996). Subjective assessments were administered on a laptop using proprietary software (Scheduled Measurement System, INC Research).

Pupil diameter was used as an objective physiological PD measure (NeurOptics Pupillometer, Irvine, Calif., USA). Measurements were collected at pre-infusion and at 5, 15, 30 and 45 minutes post-infusion and 1, 1.5, 2, 3, 4, 5 and 8 hours post-infusion under mesopic lighting conditions.

### Pharmacokinetic Assessments

At each treatment session, venous blood samples were collected at pre-infusion and at 5, 15, 30 and 45 minutes post-infusion and 1, 1.5, 2, 3, 5 and 8 hours post-infusion to determine plasma concentrations of HMO and naloxone.

### Safety Assessments

Safety and tolerability were assessed regularly through adverse events (AEs), vital signs, 12-lead electrocardiogram (ECG), continuous telemetry and clinical laboratory.

### Statistical Analysis

Pharmacodynamic analysis. For all subjective measures, the primary endpoints were Eₘₐₓ (maximum effect) or Eₘᵢₙ (minimum effect). For pupillometry measures, the primary endpoint was the maximum pupil constriction (MPC). Endpoints were analyzed using a mixed-effect model for a crossover study. The mixed-effect model had treatment, period and sequence as fixed effects, baseline (pre-infusion) measurement as a covariate, where applicable, and subject nested in the sequence as a random effect. The statistical significance of all treatment differences was reported; all statistical tests were conducted using 2-tailed significance criteria (α=0.05).

Tests for non-normality and homogeneity of variance were conducted for the primary measures (Drug Liking VAS, ARCI MBG, and pupillometry). Non-parametric sensitivity analyses were conducted, if necessary, for the primary measures. Planned contrasts were between all naloxone/HMO combinations vs. HMO/naloxone placebo. Additional pairwise comparisons were made to evaluate differences between all naloxone/HMO combinations.

To further evaluate the reduction in drug liking of naloxone/HMO combinations (test drug, T) relative to HMO alone (positive control, C), a post hoc responder analysis using percent reduction in Eₘₐₓ of Drug Liking VAS was conducted (FDA, 2013). Percent reduction in Eₘₐₓ of Drug Liking VAS was calculated as follows:

$$\% reduction = \frac{C - T}{C - 50} \times 100$$

A responder was defined as a subject who demonstrated a desired % reduction in Eₘₐₓ of Drug Liking for T relative to C. A clinically meaningful response in reduction is as yet unknown; therefore, responders were categorized into those who demonstrate a 30%, 40% or 50% reduction in Eₘₐₓ of Drug Liking. A proportion test was used to determine the statistical significance of the responder rate within each category.

Pharmacokinetic analysis. Plasma samples were analyzed for HMO and naloxone concentrations. For each subject, the following PK parameters were estimated using non-compartmental analysis: maximum plasma concentration (Cₘₐₓ), time to Cmax (Tₘₐₓ), area under the plasma concentration-time curve from time zero until the last quantifiable concentration (AUCₗₐₛₜ), AUC extrapolated to infinity (AUC_{inf}) elimination half-life (t_{1/2}), clearance (CL) and volume of distribution (V).

PK/PD analysis. Relationships between PK and PD parameters were evaluated graphically and using linear regression. Scatter plots were produced in which PD parameters were plotted against plasma concentrations for all naloxone/HMO combinations. Exploratory analyses evaluated the relationship between the objective (pupillometry) and subjective endpoints (Drug Liking VAS and EOD measures) using Pearson/Spearman correlations and regression approaches. Relationship to naloxone dose was analyzed using regression

Safety. AEs, vital signs, 12-lead ECG and clinical laboratory assessments were summarized descriptively, but not analyzed.

### Results

### Demographics and Disposition

A total of 30 qualified subjects were randomized to the treatment phase. Subjects had a mean (SD) age of 33.0 (6.3) years, were mainly male (80%), and white (83.3%) with a BMI range of 19.6 to 29.8 kg/m² and mean (SD) body weight of 75.9 (12.5) kg. All subjects had previous experience with opioids, and the majority also had experience with cannabinoids and stimulants (˜70%). A total of 26 subjects completed the study and were included in the PD analyses. Two (6.7%) subjects withdrew consent, and 2 (6.7%) subjects were discontinued for administrative reasons.

### Subjective and Physiological Effects of Hydromorphone

Hydromorphone 30 μg/kg produced subjective effects typical of opioid administration, as seen by high Eₘₐₓ on measures of drug liking, willingness to take drug again, positive effects (e.g., ARCI-MBG) and sedative effects (low Eₘᵢₙ, indicating greater drowsiness), and minimal negative effects. Mean peak scores were typically observed between 5 and 15 minutes post-infusion and scores declined steadily to near neutral levels thereafter.

Administration of HMO resulted in a sharp decrease in pupil diameter (ie, miosis) that was sustained for approximately 1 hour. Thereafter, mean pupil diameter gradually increased to near baseline levels by 8 hours post-infusion. In contrast to mean peak scores on the primary subjective measures, the mean peak decrease in pupil diameter was observed to be later, occurring at approximately 0.75 hours post-infusion.

### Effect of Naloxone on Subjective Responses to Hydromorphone

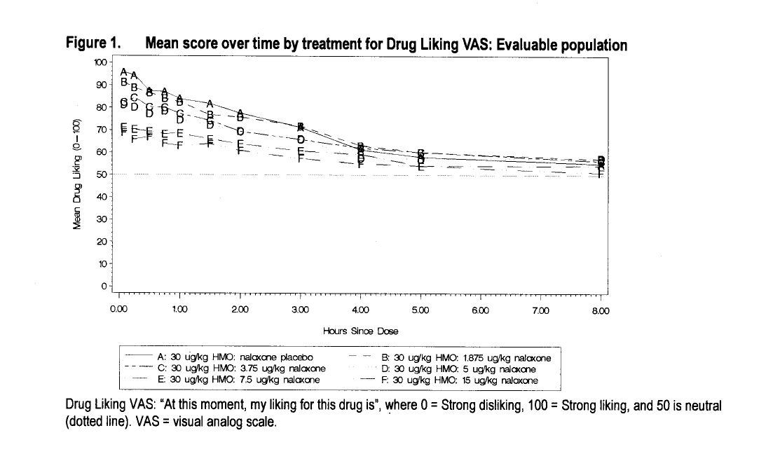

Administration of naloxone resulted in a dose-dependent decrease in mean Drug Liking VAS scores for HMO during the first 3 hours post-infusion; however, the decrease was not apparently linear, as there was a ‘clustering’ in the pattern of scores over time following administration of HMO+naloxone 3.75 μg/kg and HMO+naloxone 5 μg/kg, and scores for HMO+naloxone 7.5 μg/kg and HMO+naloxone 15 μg/kg were similar to each other in magnitude and time course—see FIG. 1.

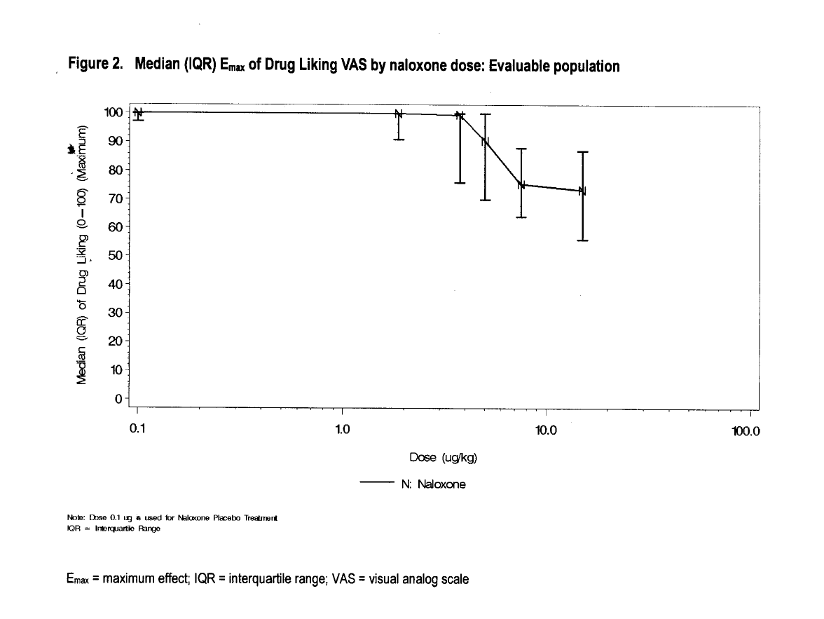

Based on the outcome of the test of normality, Drug Liking VAS parameters were analyzed non-parametrically. Median values Eₘₐₓ values for Drug Liking VAS of HMO decreased with naloxone administration; however, the decrease was not apparently linear, but showed a more step-like decrease at the two higher doses of naloxone—see FIG. 2. A main effect of treatment was observed to be statistically significant for Eₘₐₓ (P<0.0001). Pairwise comparisons revealed that administration of HMO with naloxone doses of 3.75 μg/kg or higher were associated with significantly lower Eₘₐₓ of Drug Liking compared to HMO alone. Pairwise comparisons between HMO+naloxone dose ratios showed that Eₘₐₓ of the 2 lowest dose ratios (HMO+naloxone 7.5 μg/kg and 15 μg/kg) not different from one another, but both were statistically significantly lower compared to Eₘₐₓ of the higher dose ratios tested (HMO+naloxone 1.875 μg/kg, 3.75 μg/kg, and 5 μg/kg). The 2 highest dose ratios (HMO+naloxone 1.875 μg/kg and 3.75 μg/kg) did not differ statistically on Eₘₐₓ.

In addition to the primary analysis of Drug Liking VAS Eₘₐₓ, a post hoc responder analysis was conducted to evaluate the proportion of subjects who showed a specified percent reduction in Eₘₐₓ following administration of the different doses of naloxone with HMO. The majority of subjects (69%) showed at least a 30% reduction in “at this moment” Drug Liking VAS Eₘₐₓ following administration of HMO+naloxone 7.5 μg/kg and 15 μg/kg compared to HMO alone, and 54% and 62%% showed at least a 40% reduction, respectively. The responder rate was statistically significant at the 30% reduction level (P=0.003) for both dose ratios.

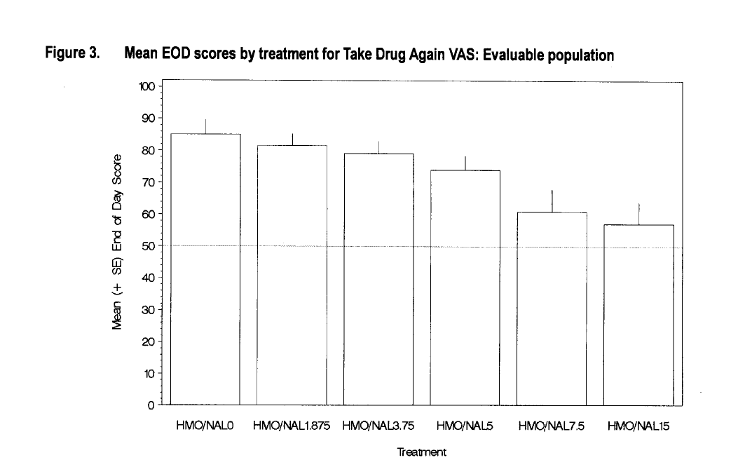

For Overall Drug Liking VAS, EOD scores were significantly lower for HMO+naloxone 7.5 μg/kg and naloxone 15 μg/kg compared to HMO alone and the higher HMO:naloxone ratios. Similarly, co-administration of HMO+naloxone doses of 5 μg/kg or higher was associated with a significantly lower EOD score on Take Drug Again VAS compared to HMO alone, with the 2 lowest ratios separating statistically from all other treatments—see FIG. 3. SDV showed a slightly different pattern, with all naloxone doses significantly reducing perceived value of HMO (alone: $29.45 [14.35]), but in general the 2 lowest ratios separated from the other treatments (HMO+naloxone 7.5 μg/kg=14.56 [13.73], +naloxone 15 μg/kg=12.07 [14.42]).

Results of positive measures (ARCI-MBG, High VAS, Good Effects VAS) showed a similar pattern of effects as the balance of effects measures with naloxone doses of 5 μg/kg or higher significantly reducing Eₘₐₓ compared to that of HMO alone. Both naloxone 7.5 μg/kg and 15 μg/kg resulted in statistically significantly lower Eₘₐₓ for HMO compared to the 2 lowest doses of naloxone (P<0.0001), but peak effects observed following administration of naloxone 7.5 μg/kg did not consistently separate from peak effects observed at 5 μg/kg. Negative effects (Bad Effects VAS, Feeling Sick VAS) were low and variable for each treatment, and no statistically significant effects were observed.

Independent of dose, co-administration of naloxone with HMO was associated with significantly lower drowsiness (Eₘᵢₙ) compared to HMO alone. Similar results were seen for Any Effects VAS, though co-administration of the 2 lowest doses of naloxone did not elicit significant reductions in Eₘₐₓ of Any Effects VAS compared to HMO alone.

### Effect of Naloxone on Physiological Response to Hydromorphone

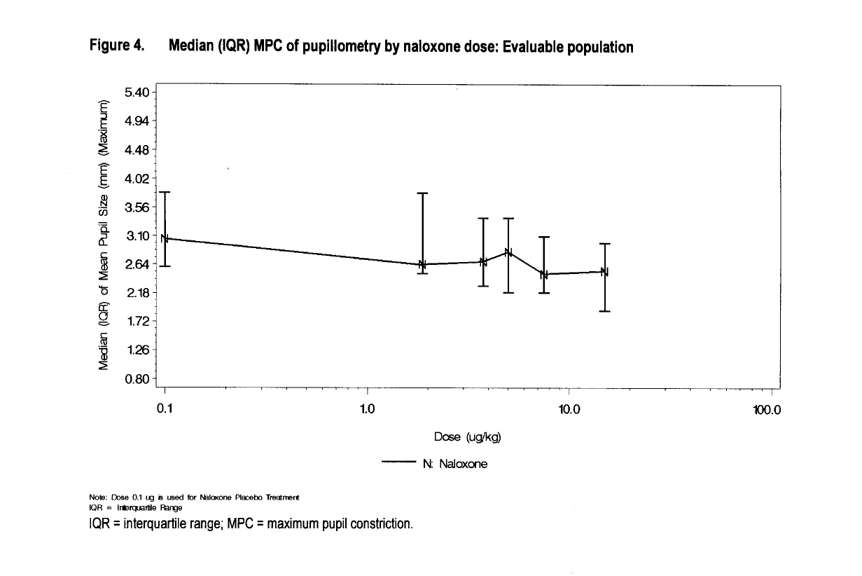

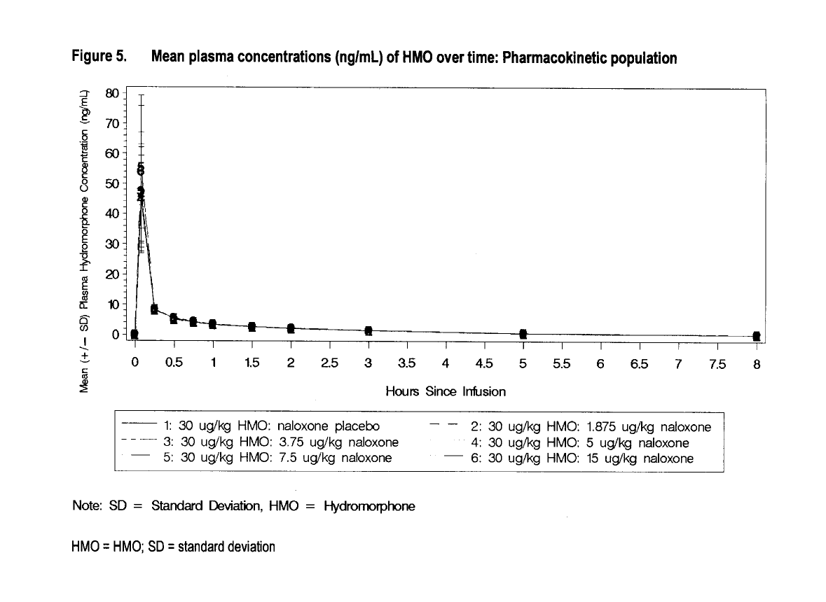

Analysis of MPC values revealed that co-administration of all tested doses of naloxone with HMO was associated with a significantly smaller MPC compared to HMO alone—see FIG. 4.

### Pharmacokinetics of Hydromorphone and Naloxone

The plasma concentration-time profile of HMO is presented in FIG. 5. Mean Cₘₐₓ of HMO was ˜45-47 ng/mL for most treatments, with the exceptions of HMO+naloxone 7.5 μg/kg and 15 μg/kg, for which mean Cₘₐₓ values were observed to be ˜54-55 ng/mL. Other derived PK parameters for HMO were similar for all treatments: mean AUCs were approximately 16 ng*hr/mL, T_{1/2}˜1.9 hours, CL ˜128 L/hr, and V ˜350-375 L. Since plasma concentrations were not sampled during and immediately following the HMO infusion, AUCs were simply calculated from the first post-infusion time point of 5 minutes and onward to avoid misestimating AUCs via back extrapolation.

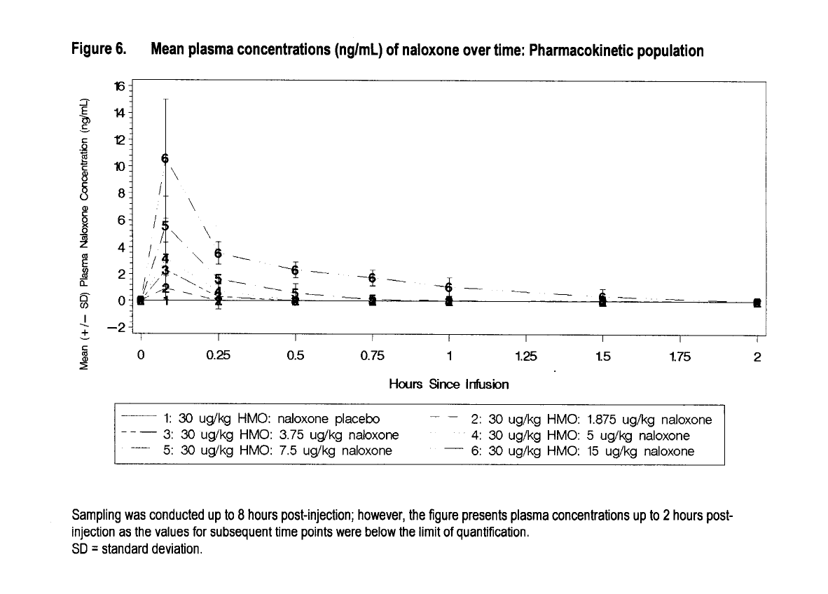

Mean plasma naloxone concentrations increased with dose. The time course was similar for each treatment, with maximum mean concentrations observed at 5 minutes post-infusion—see FIG. 6. Mean plasma naloxone concentrations decreased rapidly in a bi-exponential manner and were BLQ by 15 minutes (naloxone 1.875 μg/kg) to 2 hours (naloxone 15 μg/kg) post-injection.

### PK/PD Analysis

The relationship between co-administered naloxone dose and the response on subjective measures and pupillometry was analyzed using linear regression. For peak or EOD scores on subjective measures of positive and other effects, regression analyses were statistically significant; results of negative effects measures were not significant, consistent with the ANOVA. Although statistically significant, the relationship between these measures and co-administered naloxone dose was relatively weak (R²=0.10-0.22).

In contrast with the subjective measures, the physiological endpoints, MPC and (pupillary area under the curve) PAOC, showed a much stronger positive relationship with naloxone dose, as shown by R² values of ˜0.79 and 0.67, respectively.

The relationship between naloxone Cₘₐₓ and peak response on the subjective and objective measures was evaluated using linear regression. Higher mean plasma concentrations of naloxone were associated with lower peak responses on the subjective measures, supporting that increasing plasma concentrations of naloxone are associated with greater antagonism of HMO effects. The relationship was significant for 6 of the 8 subjective measures, though the correlations were weak (R²=0.054-0.175). In contrast with the strong relationship between dose and MPC, the correlation between Cₘₐₓ and MPC was not statistically significant. Miosis was seen to be significantly lower following all naloxone treatments, including HMO+naloxone 1.875 μg/kg (MPC=2.55 mm), compared to HMO alone (3.05 mm), with decreases in MPC only slightly larger following administration of the higher naloxone doses (maximum change from HMO alone was 0.56 mm) Also, MPC was observed to be later (0.75-1.5 hours post-infusion) than the Cₘₐₓ, suggesting that concentrations at the effect site follows a complex relationship with plasma concentration and likely explains the absence of a correlation between the 2 parameters.

### Safety

Overall, all treatments were well-tolerated. There were no deaths or other Serious Adverse Events (SAEs), and no subject was discontinued from the treatment phase because of a Treatment Emergent Adverse Event (TEAE). Most subjects had TEAEs that were at most mild in severity, and 10 subjects experienced a TEAE of moderate severity. The most common TEAE following administration of HMO alone was euphoric mood (73.3%). The incidence of euphoric mood was considerably lower following co-administration of HMO with naloxone 5 μg/kg (57.1%), 7.5 μg/kg (44.4%), and 15 μg/kg (32.1%). Following administration of HMO alone, the incidence of more common side effects of opioid administration were somnolence (60.0%), feeling hot (46.7%), pruritus (40.0%), blurred vision (10.0%), and vomiting (13.3%). When co-administered with naloxone doses of 5 μg/kg and higher, the incidence of such TEAEs showed notable decreases, similar to euphoric mood.

All mean clinical laboratory, vital signs and ECG values were within the normal ranges, and no subject experienced clinically significant changes in laboratory or ECG values during the study. With the exception of 3 spontaneous TEAEs of decreased respiratory rate and 1 spontaneously reported event of respiratory depression, which all occurred within 1 minute of dosing and resolved without intervention, no individual out-of-range values collected at the scheduled time points were clinically significant.

### Discussion and Conclusions

Co-administration of naloxone significantly reduced liking and the euphoric effects of HMO, in addition to the sedative and miotic effects of HMO. Based on convergence of the subjective and objective measures data, both 4:1 and 2:1 ratios significantly and consistently reduced the intravenous abuse potential of HMO.

In choosing a dose ratio that will reduce the potential for intravenous abuse, the selected dose of naloxone needs to be balanced against potential negative effects on analgesic efficacy of the oral formulation in the intended patient population. Although oral bioavailability of naloxone is very low (<3%), it is variable and there is a potential for reduced analgesia at high enough doses. Therefore, a range of naloxone doses less than 15 μg/kg was also evaluated. A dose-proportional decrease in subjective effects might be expected with increasing dose of naloxone, but comparisons across treatments showed a stepwise, rather than linear, reduction on measures of liking and other euphoric effects (e.g., ARCI-MBG) between the higher dose ratios (>6:1) and the 4:1 and 2:1 ratios, which were not statistically different from each other. The latter observation may also suggest a plateau was reached, and that higher ratios would not necessarily provide additional meaningful reductions. Importantly, the observed pattern of effects cannot be readily attributable to exposure levels of naloxone.

Taken together, the convergence of subjective and objective PD results and the safety findings demonstrate that the HMO:naloxone ratio of 4:1 and 2:1 would be suitable for use in a combination HMO analgesic product that would deter high risk intravenous abuse. It is believed that such a product could confer important public health benefits.

### Example 2

In this example, there is described a randomized, double-blind, dose-ranging crossover study evaluating the effect of naloxone on intravenous hydromorphone (HMO) abuse potential in opioid dependent drug users.

### Overall Design

This was a single-centre, double-blind, randomized, crossover dose-ranging study to identify the intravenous (IV) abuse potential, PD, and physiologic effects of HMO administered with naloxone compared with HMO alone in opioid-dependent subjects. The study consisted of a standard medical screening visit, HMO dose selection phase, which was used to identify an appropriate HMO test dose to be used for the duration of the study, HMO dose stabilization phase, treatment phase and end-of-study phase. All subjects were offered counseling services and referral to treatment while they were in this study and subjects were required to meet with an addiction counselor at least once during their stay in clinic.

### Participants

Eligible subjects were opioid-dependent male and female adult volunteers, between 18 and 55 years of age, inclusive, with a body mass index (BMI) between 18.0 and 33.0 kg/m², inclusive. Subjects were self-reported regular opioid users who had experience with IV opioid administration for the purpose of misuse/abuse and who were able to tolerate daily IV opioid administration. Subjects had to meet current drug dependence criteria as defined by DSM-IV and included subjects who had previously been in a drug rehabilitation program.

### Dose Selection and Dose Stabilization

The purpose of this two-day double-blind, placebo-controlled two-way crossover dose selection phase and the one day dose stabilization phase was to identify an appropriate personalized test dose for each individual subject. Test-doses were selected based on subjects self-reported opioid use history. During the dose selection phase, subjects were required to demonstrate an appropriate response to placebo, and discriminate the effects of a single intravenous dose of HMO compared to placebo in a laboratory setting. Specifically, subjects had to achieve a peak response (Eₘₐₓ) to HMO that was at least 20% greater than that of placebo on “at this moment” Drug Liking visual analog scale (VAS) and show an acceptable placebo response, defined as a VAS response between 40 to 60 inclusive for Drug Liking.

Subjects were given two opportunities to pass the dose selection phase (Day 1 and Day 2) to ensure that the appropriate test dose was identified. Subjects also underwent a mandatory one or two day dose stabilization phase in which they received two oral maintenance doses of HMO to ensure that they did not experience withdrawal effects prior to the administration of naloxone. The oral maintenance dose was initially equivalent to or less than the test dose, but adjustments were allowed based on safety information and investigator discretion.

### Treatment Phase

The treatment phase consisted of 5 consecutive dosing days in which subjects received 4 HMO+naloxone doses and 1 dose of HMO+naloxone placebo (HMO alone; naloxone placebo administered as saline infusion). The HMO+naloxone combination doses were administered in the following fixed order: 8:1, 6:1, 4:1, and 2:1. The HMO alone arm was randomized within the HMO+naloxone doses in order to maintain blinding. Subjects received an oral HMO maintenance dose approximately 8 hours following administration of the test dose.

A rescue protocol was implemented for instances of withdrawal prior to administration of the maintenance dose. This protocol permitted subjects exhibiting withdrawal symptoms to receive additional doses of oral immediate-release HMO (doses of 1 mg to 4 mg up to 12 mg/daily) or IV HMO following treatment dose administration (doses of up to 4 mg per injection), as needed based on OOWS and SOWS scores and investigator clinical judgement. Pharmacodynamic assessments were conducted up to 8 hours post dose (prior to administration of maintenance dose) and safety measurements were conducted up to 12 hours post dose.

### End of Study Phase

Subjects were discharged on the morning following the last day of dosing. Prior to discharge, subjects underwent End-of-Study procedures and were administered an optional final morning maintenance dose of oral HMO. Subjects were discharged once considered medically stable (a minimum of 2 hours after their final dose of HMO).

### Pharmacodynamic Assessments

Opioid withdrawal associated with naloxone administration was assessed using the Objective Opioid Withdrawal Scale (OOWS) and the Subjective Opioid Withdrawal Scale (SOWS). These were administered at pre-infusion, 5 and 30 minutes post-infusion and 1, 2, 3, 4, 6 and 8 hours post-infusion. Both scales were also administered as needed to determine whether rescue dosing was required. In the OOWS, an independent observer rated as present or absent each of 13 physically observable signs or behaviours associated with opioid withdrawal based on a 5-minute period of observation (score range is 0-13). In the SOWS, subjects rated the intensity of each of 16 common motoric, autonomic, gastrointestinal, musculoskeletal, and psychic symptoms of opioid withdrawal on a 5-point Likert scale, i.e., from 0 (“not at all”) to 4 (“extremely”), based on how they were feeling at the time of testing (score range is 0-64).

‘At this moment’ subjective measures VAS of Drug Liking (bipolar), High, Good Drug Effects, Bad Drug Effects, Nausea, Feeling Sick, Anxiety re: Drug Effects VAS, and Any Effects VAS were administered at pre-infusion (measures assessing specific drug effects were not administered at pre-infusion) and at 5, 15, 30 and 45 minutes post-infusion and 1, 1.5, 2, 3, 4, 6 and 8 hours post-infusion. Overall Drug Liking VAS and Take Drug Again VAS are global measures that were administered at 4 and 8 hours post-infusion. Each unipolar VAS was scored as an integer from 0 to 100, with anchors such as “Not at all” (score=0) to “Extremely” (score=100). Bipolar scales of Drug Liking used anchors “Strong Disliking” (score=0) and “Strong Liking” (score=100), with a score of 50 being neutral. Subjective assessments were administered on a laptop using proprietary software (Scheduled Measurement System, INC Research).

Pupil diameter was used as an objective physiological PD measure (NeurOptics Pupillometer, Irvine, Calif., USA). Measurements were collected at pre-infusion and at 5, 15, 30 and 45 minutes post-infusion and 1, 1.5, 2, 3, 4, 6 and 8 hours post-infusion under mesopic lighting conditions.

### Safety Assessments

Safety and tolerability were assessed regularly through adverse events (AEs), vital signs, 12-lead electrocardiogram (ECG), continuous telemetry and clinical laboratory measurements.

### Subject Questionnaires

Two voluntary questionnaires were included within the study design. The Refusal to Participate Questionnaire was administered to those subjects who declined participation in the study. Subjects were questioned on their reason for refusal.

In order to collect additional information on subject opioid use and tampering history, and subjects' perception of agonist/antagonist combination products, including what properties of the product might have deterred subjects from enrolling in the study, an optional questionnaire was administered to all subjects who came in for Screening (whether they completed the Screening Phase or not). The Perception of Opioid/Naloxone Combination Product Questionnaire consisted of 9 questions.

### Statistical Analysis

Pharmacodynamic Analysis. For all subjective measures and the OOWS and SOWS, the primary endpoints were Eₘₐₓ (maximum effect) or Eₘᵢₙ (minimum effect). For pupillometry measures, the primary endpoint was the maximum pupil constriction (MPC). Rescue medication and withdrawal endpoints (study discontinuation) were summarized using descriptive statistics.

Endpoints were analyzed using a mixed-effect model for a crossover study. The mixed-effect model had treatment, period and sequence as fixed effects, baseline (pre-infusion) measurement as a covariate, where applicable, and subject nested in the sequence as a random effect. The statistical significance of all treatment differences was reported; all statistical tests were conducted using 2-tailed significance criteria (α=0.05). Tests for non-normality and homogeneity of variance were conducted for all measures. Non-parametric sensitivity analyses were conducted, if necessary. Planned contrasts were between all naloxone/HMO combinations vs. HMO/naloxone placebo. Additional pairwise comparisons were made to evaluate differences between all naloxone/HMO combinations.

Safety Analysis. AEs, vital signs, 12-lead ECG and clinical laboratory assessments were summarized descriptively, but not analyzed.

Analysis of Subject Questionnaires. Results of the Refusal to Participate Questionnaire and the Perception of Opioid/Naloxone Combination Product Questionnaire were summarized using all available data by incidence of responses to each question.

### Results

### Demographics and Disposition

A total of 67 subjects were screened for the study. Of those, 22 subjects were eligible to proceed to the HMO dose selection phase. Twelve (12) subjects continued on to the dose stabilization phase and were considered eligible for the treatment phase. Randomized subjects had a mean (SD) age of 34.5 (9.8) years, were mainly male (91.7%), and were all white (100%) with a BMI range of 20.5 to 30.7 kg/m² and mean (SD) body weight of 73.1 (13.9) kg. All subjects were opioid-dependent. Of the 12 randomized subjects, 12 (100%) subjects received at least one dose of study drug and 7 (58.3%) subjects received all 5 study treatments.

Five subjects (41.7%) withdrew early from the treatment phase, 2 (16.7%) subjects withdrew early due to treatment-emergent AEs (TEAEs) following treatment with HMO+naloxone 6:1, 3 subjects withdrew consent for personal reasons, 1 following treatment with HMO+naloxone placebo, 1 following treatment with HMO+naloxone 6:1, and 1 following treatment with HMO+naloxone 4:1.

### Subjective and Physiological Effects of Hydromorphone

Administration of hydromorphone alone produced subjective effects typical of opioid administration, as seen by high Eₘₐₓ on measures of drug liking, willingness to take drug again, and positive effects. Pupil diameter and subjective withdrawal (SOWS) scores were lower following administration of HMO alone. The effects of HMO alone had a rapid onset following IV administration, with peak effects observed within 5 to 15 minutes of dosing.

### Effect of Naloxone on Opioid Withdrawal Measures

Co-administration of naloxone with HMO was associated with statistically significant increases in self-reported and objectively rated opioid withdrawal effects (primary endpoints).

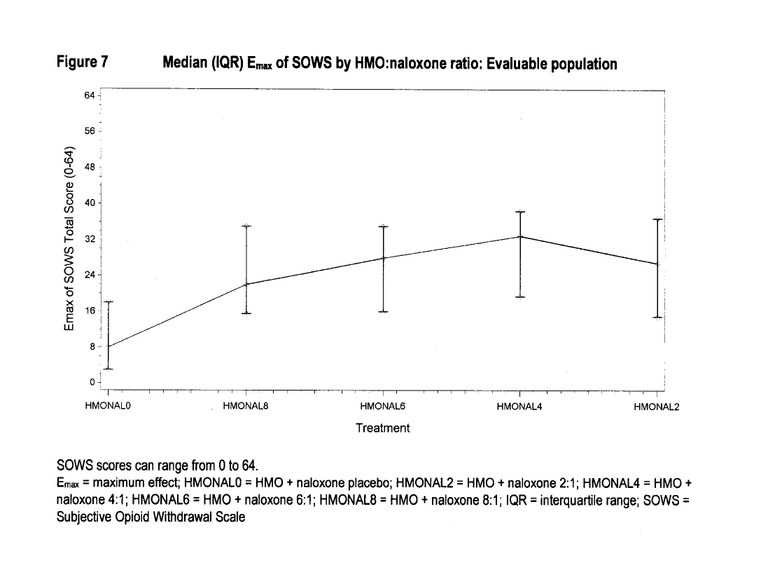

For SOWS, HMO+naloxone 8:1 showed little change from pre-infusion, while HMO+naloxone 6:1, 4:1, and 2:1 were associated with increased scores relative to baseline, which peaked at 5 minutes post-infusion. HMO+naloxone 2:1 was associated with the highest scores and this effect was sustained over the first 30 minutes post-infusion. HMO+naloxone 2:1 also showed the highest mean Eₘₐₓ, while the highest median Eₘₐₓ was observed with the HMO+naloxone 4:1 treatment (FIG. 7).

Based on the outcome of the test of normality, SOWS Eₘₐₓ was analyzed non-parametrically. There was a significant main effect of treatment for SOWS Emax (P=0.034). Pairwise comparisons showed statistically significant differences between HMO+naloxone 8:1, 6:1, and 4:1 compared to HMO alone. A trend toward a statistically significant difference between HMO+naloxone 2:1 and HMO alone (P=0.063) was observed, possibly due to the smaller sample size and greater variability (IQR: 0.0-24.0) seen in the 2:1 treatment group. The smaller sample size in the 2:1 ratio was a result of subjects withdrawing before reaching the 2:1 ratio due to intolerable withdrawal effects at lower levels ratios of naloxone. There were no significant differences between the different HMO+naloxone ratios on this endpoint.

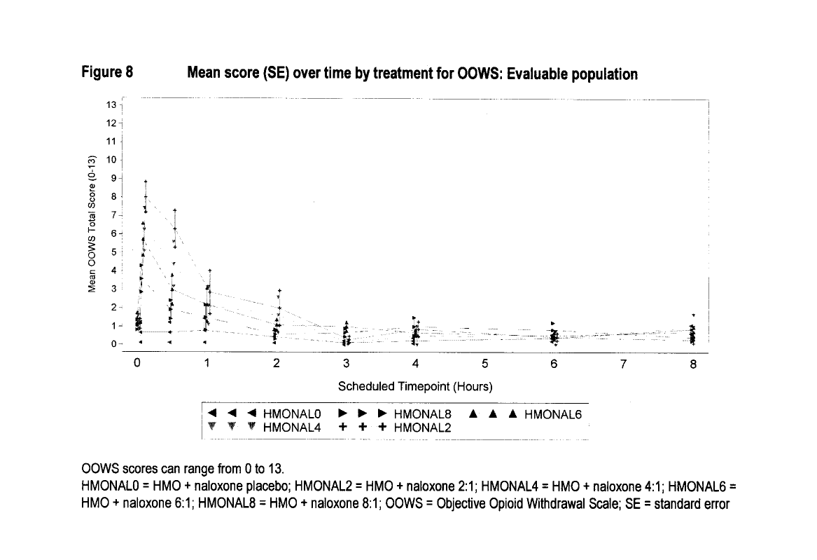

OOWS scores over time showed an orderly increase with increasing HMO:naloxone ratio. Independent of ratio, effects of HMO+naloxone treatments on this measure peaked at 5 minutes post-infusion and lasted for approximately 2-3 hours (FIG. 8). Consistent with the time course profiles, mean and median OOWS Eₘₐₓ increased with increasing HMO:naloxone ratio; all HMO+naloxone treatments were associated with higher values relative to HMO alone. A significant overall treatment effect was observed with OOWS Eₘₐₓ (P<0.001). Pairwise comparisons revealed significant differences between all 4 HMO+naloxone treatments and HMO alone.

### Effect of Naloxone on Subjective Responses to Hydromorphone

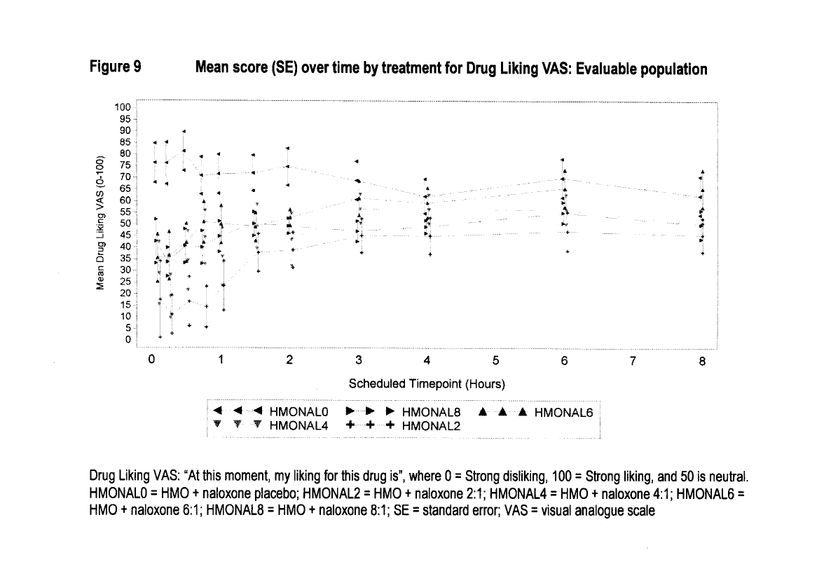

Drug Liking VAS scores for all HMO+naloxone treatments were much lower than for HMO alone, with scores falling below neutral and in the “disliking” range (<50) from immediately following dosing (5 minutes) until between 45 minutes to 2 hours post-infusion. Scores were lowest following administration of HMO+naloxone 2:1, followed by HMO+naloxone 4:1, while the patterns of scores for HMO+naloxone 6:1 and 8:1 were similar (FIG. 9).

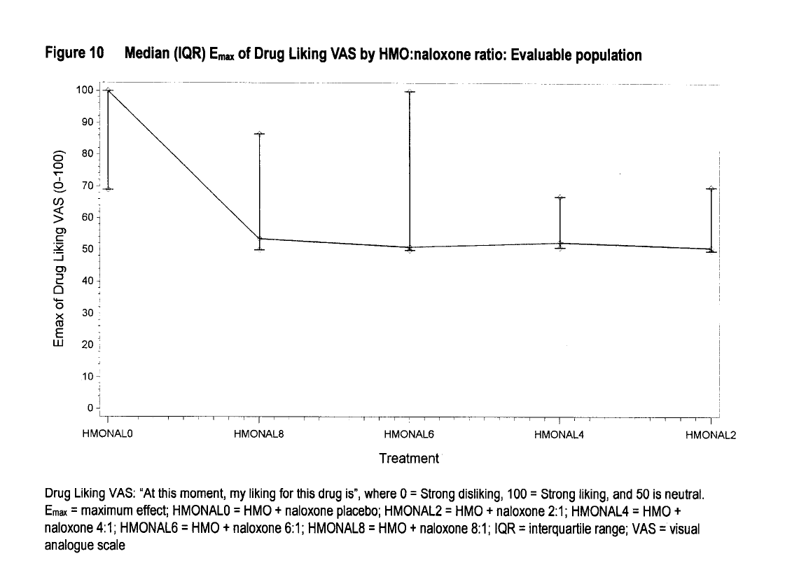

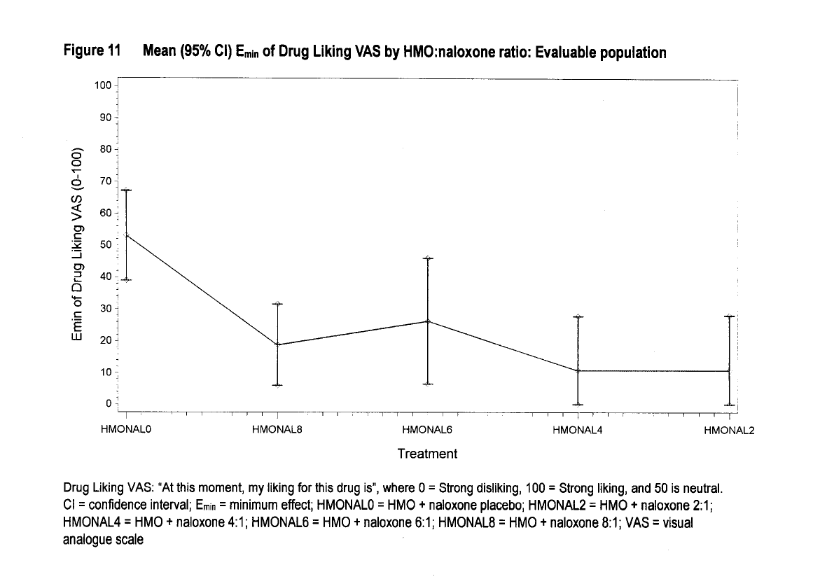

Mean Drug Liking VAS Eₘₐₓ values decreased with increasing naloxone dose, with the highest scores observed for HMO+naloxone 8:1 and HMO+naloxone 6:1 followed by the HMO+naloxone 4:1 and HMO+naloxone 2:1 ratios. However, median Eₘₐₓ scores for all HMO+naloxone treatments were lower and similar across doses, hovering around the neutral mark (50; FIG. 10). Eₘᵢₙ values (e.g., maximum “disliking”) for HMO+naloxone treatments were markedly lower compared to HMO alone. HMO+naloxone 4:1 and HMO+naloxone 2:1 had the lowest mean and median Eₘᵢₙ values (median of 0.0 for both), followed by HMO+naloxone 8:1 and HMO+naloxone 6:1 (FIG. 11).

Based on the outcome of the test of normality, Drug Liking VAS Eₘₐₓ was analyzed non-parametrically and Eₘᵢₙ was analyzed using parametric statistics. An overall treatment effect was observed to be statistically significant for Eₘₐₓ, and Eₘᵢₙ, (P<0.01 for all). Pairwise comparisons revealed that administration of HMO+naloxone at the 8:1, 4:1, and 2:1 dose ratios was associated with significantly lower Eₘₐₓ compared to HMO alone. Eₘₐₓ for HMO+naloxone 6:1 was not statistically different from HMO alone. Pairwise comparisons between HMO+naloxone dose ratios did not reveal any significant differences between the different HMO:naloxone dose ratios. Similar results were seen for Eₘᵢₙ; however, for Eₘₘ, significant differences were also seen between the HMO:naloxone dose ratios. Eₘᵢₙ of HMO+naloxone 8:1 had a significantly lower Eₘᵢₙ value (less disliking) in comparison to HMO+naloxone 6:1.

For balance of effects measures (Overall Drug Liking and Take Drug Again VAS), when compared to HMO alone, end of day scores were significantly lower for HMO+naloxone at ratios of 4:1 and 2:1, but were not significantly lower at ratios of 8:1 or 6:1.

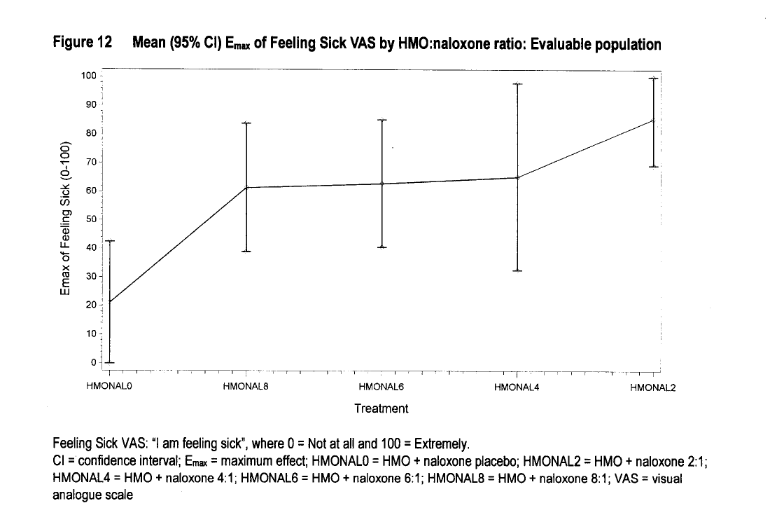

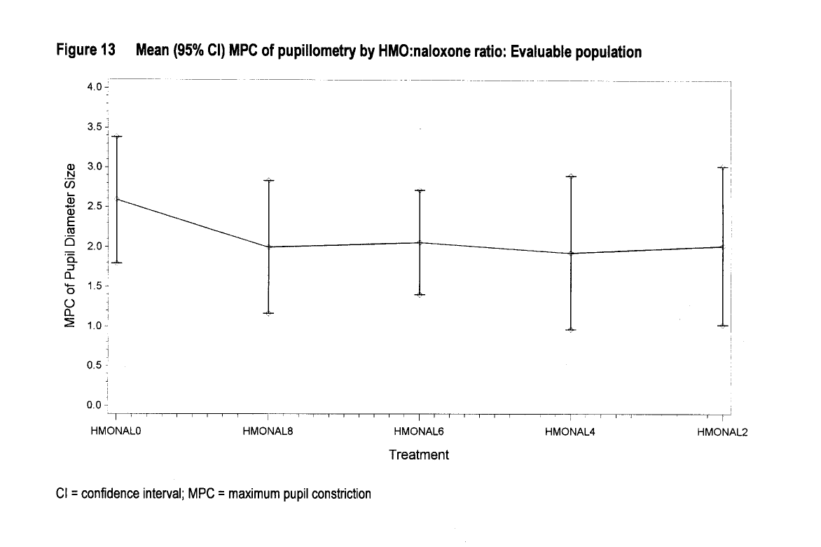

In general, positive effects of HMO were significantly lowered with co-administration of naloxone. The 6:1 and 2:1 HMO+naloxone treatments showed significantly lower scores on High VAS and the 8:1 and 6:1 HMO+naloxone treatments showed significantly lower scores on Good Effects VAS when compared to HMO alone. Co-administration of naloxone was associated with significant increases in negative effects for all dose ratios. With the exception of the 6:1 ratio, all naloxone doses were associated with significantly higher scores on the Bad Effects and Feeling Sick VASs (FIG. 12). Anxiety related to treatment dosing was also significantly greater for all HMO+naloxone treatments in comparison to HMO alone.

### Effect of Naloxone on Physiological Response to Hydromorphone

Analysis of MPC values revealed that co-administration of naloxone antagonized HMO-induced miosis, as measured by statistically significant differences in MPC and PAOC with all HMO+naloxone treatments; however, there were no significant differences between naloxone dose ratios.

### Effect of Naloxone on Objective Measures

During the treatment phase, rescue medication was administered as needed following administration of a treatment dose. Oral and IV rescue medication were administered in doses of 1 to 4 mg. Most subjects who received at least one dose of the HMO+naloxone treatment required oral or IV rescue medication within 10 to 20 minutes of treatment administration. In most subjects, oral doses were less effective for rapidly treating severe withdrawal; therefore, oral rescue HMO was not administered as often as IV HMO. The average dose and maximum dose of oral rescue medication was similar across treatments, ranging between 1 and 2 mg of oral HMO. Similarly, the mean number of oral rescue administrations and the mean total dose administered did not differ substantially between 8:1, 6:1, and 4:1 HMO+naloxone.

In general, a larger number of subjects received IV rescue medication after administration of the HMO+naloxone 4:1 and 2:1. The highest total dose of IV rescue medication was administered following administration of the 6:1 HMO+naloxone dose. For most subjects, these IV rescue medication doses were separated into 2 to 5 administrations. An examination of individual subject total rescue medication dose suggests that, across all subjects, the dose of rescue medication required increased with higher doses of naloxone.

### Perception of Opioid/Naloxone Combination Product Questionnaire

In total, 72 subjects completed the questionnaire. Results of the questionnaire indicated that subjects had experience with a wide variety of opioid products (oxycodone+acetaminophen (Percocet®), heroin, other oxycodone products, immediate-release HMO, immediate-release oxycodone, controlled-release morphine, OxyNEO®, and Tylenol 3s/4s or other codeine products). The most common method of administration was snorting, followed by swallowing the drug whole, injection-extracted from a tablet or capsule, and injection with an IV formulation. The most common tampering method was crushing followed by removing coating or layers, dissolving in solution for injection, heating/melting/boiling, and chewing. Only a small number of subjects reported experience with opioid agonist/antagonist combination products. Most subjects reported that they had not had the opportunity to try a combination product, and most reported not knowing about the risk of withdrawal with combination products.

### Safety

With the exception of expected opioid withdrawal effects, all treatments were relatively well tolerated. There were no deaths or other SAEs. The incidence of TEAEs was highest with HMO+naloxone 2:1 (85.7%), followed by HMO+naloxone 6:1 (81.8%), HMO alone (77.8%), and HMO+naloxone 8:1 and HMO+naloxone 4:1 (75.0% each). Although none of the subjects voluntarily withdrew from the study as a result of drug withdrawal effects, 2 subjects were withdrawn from the study due to moderate TEAEs of drug withdrawal syndrome following administration of HMO+naloxone 6:1. One subject experienced a severe TEAE of drug withdrawal syndrome after receiving HMO+naloxone 2:1. The most common TEAEs following administration of HMO alone were euphoric mood (33.3%), pruritus (11.1%), and feeling hot (22.2%)—all consistent with the known pharmacology of HMO. The incidence of these opioid side effects decreased (HMO+naloxone 8:1) or did not occur (HMO+naloxone 6:1, 4:1, and 2:1) following co-administration of HMO+naloxone. The incidence of TEAEs potentially associated with opioid withdrawal (i.e., hyperhidrosis, piloerection, cold sweat, feeling cold, drug withdrawal syndrome, chills, muscle twitching, and yawning) was higher with decreasing ratio of HMO:naloxone.

All mean clinical laboratory values and ECG results were generally within normal range, and no clinically significant findings based on these safety assessments were reported. A total of 3 subjects experienced mild TEAEs of heart rate increased following administration of HMO+naloxone. However, only one of the subjects experienced increased heart rate immediately (<1 minute) following dosing with the first HMO+naloxone dose (8:1), the timing of which suggests that it may have been associated with withdrawal-related anxiety.

### DISCUSSION AND CONCLUSIONS

In this study, the subjective and objective effects of a total of 4 HMO:naloxone ratios, ranging from 8:1 to 2:1, were evaluated and compared with those induced by HMO alone. Statistically significant differences from HMO alone were observed on “at this moment” and global measures of positive effects, demonstrating that naloxone significantly reduced the positive effects of HMO. Statistically significant effects were also observed on measures of negative effects, e.g., Bad Effects, Feeling Sick, Anxiety, demonstrating that in opioid-dependent subjects, naloxone not only reduced the positive effects, but also significantly increased the negative effects that subjects experienced. In addition, despite frequent administration of rescue medication, naloxone administration induced mild to severe withdrawal effects in most subjects, typically with more severe withdrawal effects at the higher naloxone doses.

Despite the small sample size, the data were congruent; results were consistent across all primary and secondary endpoints and were in the expected direction, suggesting that the study was successful despite the design challenges. The study results were also consistent with those reported in Example 1 conducted in non-dependent opioid users, which suggested that both 4:1 and 2:1 ratios significantly reduced the IV abuse potential of HMO. In this Example, co-administration of naloxone resulted in significantly lower scores for only the 4:1 and 2:1 ratios on balance of effects measures in comparison to HMO alone.

For the most part, significant differences among the different dose ratios were not observed, likely due to the sample size and variability in subject responses. Mean subjective responses to HMO with and without naloxone co-administration varied in this population. The inter-subject variability is likely attributable to the variation in individual doses and the potential masking of naloxone effect as a result of co-administration of rescue medication. Although, statistical differences between the different dose ratios were not consistently observed, the general pattern of results suggests that opioid-dependent users are extremely sensitive to the antagonistic effect of naloxone when co-administered with HMO and the 4:1 and 2:1 ratios appear to produce the most consistent effects.

Taken together, the convergence of subjective and objective PD results and the safety findings demonstrate that the HMO:naloxone administered in a 4:1 or a 2:1 ratio is suitable for use in a combination HMO analgesic product to deter high-risk IV abuse. It is believed that such a product would confer important public health benefits by helping to reduce high-risk abuse of prescription opioids, while continuing to provide critical pain relief.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

The following is a list of full citations for publications referred in the present specification:
Bargagli A M, Hickman M, Davoli M et al. Drug-related mortality and its impact on adult mortality in eight European countries. European Journal of Public Health 2006 April; 16:198-202.
Substance Abuse and Mental Health Services Administration. Drug Abuse Warning Network, 2007: National Estimates of Drug-Related Emergency Department Visits, Office of Applied Studies, 2010.
Griffiths R R, Rush C R, Puhala K A. Validation of the multiple-choice procedure for investigating drug reinforcement in humans. Exp Clin Psychopharmacol 1996; 4:97-106.
Griffiths R R, Troisi J R, Silverman K, Mumford G K. Multiple-choice procedure: an efficient approach for investigating drug reinforcement in humans. Behav Pharmacol 1993 February; 4(1):3-13.
Handelsman L, Cochrane K J, Aornson M J et al. Two new rating scales for opiate withdrawal. Am J Alc Abuse 1987; 13:293-308.
Hays L, Kirsh K L, Passik S D. Seeking drug treatment for Oxycontin [sic] abuse: A chart review of consecutive admissions to a substance abuse treatment facility in Kentucky. J Natl Comp Cancer Network 2003; 1(3):423-8.
Health Canada: Advisory Committee on Population Health (ACPH). Reducing the Harm Associated with Injection Drug Use in Canada. September 2001.
Martin W R, Sloan J W, Sapira J D, Jasinski D R. Physiologic, subjective, and behavioral effects of amphetamine, methamphetamine, ephedrine, phenmetrazine, and methylphenidate in man. Clin Pharmacol Ther 1971 March; 12(2):245-58.
Oliveto A H, Rosen M I, Kosten T A, Hameedi F A, Woods S W, Kosten T R. Hydromorphone-naloxone combinations in opioid-dependent humans under a naloxone novel-response discrimination procedure. Exp Clin Psychopharmacol 1998 May; 6(2):169-78.
Sadock B J, Sadock V A, Sussman N. Kaplan and Sadock's pocket handbook of psychiatric drug treatment. 4th ed. Lippincott Williams and Wilkins; 2005.
Walsh S L, Sullivan J T, Preston K L, Garner J E, Bigelow G E. Effects of naltrexone on response to intravenous cocaine, hydromorphone and their combination in humans. J Pharmacol Exp Ther 1996 November; 279(2):524-38.

What is claimed is:

1. A method of reducing drug liking a subject comprising the step of administering to the subject an oral pharmaceutical composition comprising: (i) hydromorphone or a pharmaceutically acceptable salt thereof, and (ii) naloxone or a pharmaceutically acceptable salt thereof, wherein the oral pharmaceutical composition comprising (i) and (ii) in a weight ratio equal to or less than about 4:1.

2. The method defined in claim 1, wherein the oral pharmaceutical composition comprises (i) and (ii) in a weight ratio in the range of from about 4:1 to about 1:1.

3. The method defined in claim 1, wherein the oral pharmaceutical composition comprises (i) and (ii) in a weight ratio in the range of from about 4:1 to about 1:1.5.

4. The method defined in claim 1, wherein the oral pharmaceutical composition comprises (i) and (ii) in a weight ratio in the range of from about 3.5:1 to about 1:1.5.

5. The method defined in claim 1, wherein the oral pharmaceutical composition comprises (i) and (ii) in a weight ratio in the range of from about 3:1 to about 1:1.5.

6. The method defined in claim 1, wherein the oral pharmaceutical composition comprises (i) and (ii) in a weight ratio in the range of from about 2.5:1 to about 1:1.5.

7. The method defined in claim 1, wherein the oral pharmaceutical composition comprises (i) and (ii) in a weight ratio of about 2:1.

8. The method defined in any one of claims 1-7, wherein the subject is a drug user.

9. The method defined in any one of claims 1-7, wherein the subject is an opiod drug user.

10. The method defined in any one of claims 1-7, wherein the subject is a recreational drug user.

11. The method defined in any one of claims 1-7, wherein the subject is a recreational opioid user.

12. The method defined in any one of claims 1-7, wherein the subject is a drug user who took an opioid for non-therapeutic purposes on at least 10 occasions during the 12 month period prior to the step of administering to the subject the oral pharmaceutical composition.

13. The method defined in any one of claims 1-7, wherein the subject is a dependent drug user.

14. The method defined in any one of claims 1-7, wherein the subject is an opioid-dependent drug user.

15. Use of an oral pharmaceutical composition comprising: (i) hydromorphone or a pharmaceutically acceptable salt thereof, and (ii) naloxone or a pharmaceutically acceptable salt thereof, wherein the oral pharmaceutical composition comprising (i) and (ii) in a weight ratio equal to or less than about 4:1, for reducing drug liking in a subject.

16. The use defined in claim 15, wherein the oral pharmaceutical composition comprises (i) and (ii) in a weight ratio in the range of from about 4:1 to about 1:1.

17. The use defined in claim 15, wherein the oral pharmaceutical composition comprises (i) and (ii) in a weight ratio in the range of from about 4:1 to about 1:1.5.

18. The use defined in claim 15, wherein the oral pharmaceutical composition comprises (i) and (ii) in a weight ratio in the range of from about 3.5:1 to about 1:1.5.

19. The use defined in claim 15, wherein the oral pharmaceutical composition comprises (i) and (ii) in a weight ratio in the range of from about 3:1 to about 1:1.5.

20. The use defined in claim 15, wherein the oral pharmaceutical composition comprises (i) and (ii) in a weight ratio in the range of from about 2.5:1 to about 1:1.5.

21. The use defined in claim 15, wherein the oral pharmaceutical composition comprises (i) and (ii) in a weight ratio of about 2:1.

22. The use defined in any one of claims 15-21, wherein the subject is a drug user.

23. The use defined in any one of claims 15-21, wherein the subject is an opiod drug user.

24. The use defined in any one of claims 15-21, wherein the subject is a recreational drug user.

25. The use defined in any one of claims 15-21, wherein the subject is a recreational opioid user.

26. The use defined in any one of claims 15-21, wherein the subject is a drug user who took an opioid for non-therapeutic purposes on at least 10 occasions during the 12 month period prior to the step of administering to the subject the oral pharmaceutical composition.

27. The use defined in any one of claims 15-21, wherein the subject is a dependent drug user.

28. The use defined in any one of claims 15-21, wherein the subject is an opioid-dependent drug user.

29. The method defined in any one of claims 1-14 or the use defined in any one of claims 15-28, wherein (i) is a pharmaceutically acceptable salt of hydromorphone.

30. The method defined in any one of claims 1-14 or the use defined in any one of claims 15-28, wherein (i) is hydromorphone hydrochloride.

31. The method or use defined in any one of claims 1-30, wherein (ii) is a pharmaceutically acceptable salt of naloxone.

32. The method or use defined in any one of claims 1-30, wherein (i) is naloxone hydrochloride.

33. The method or use defined in any one of claims 1-32, wherein the oral pharmaceutical composition comprises a prolonged release pharmaceutical composition.

34. The method or use defined in claim 33, wherein the prolonged release pharmaceutical composition comprises a prolonged release pharmaceutical dosage form comprising a plurality of coated beads, each of the coated beads comprising:
(a) a granule;
(b) a first layer coated on the granule, the first layer comprising: (i) hydromorphone or a pharmaceutically acceptable salt thereof, (ii) naloxone or a pharmaceutically acceptable salt thereof, (iii) an antioxidant compound, and (iii) a chelating compound; and
(c) a second layer coated on the first layer, the second layer comprising a prolonged release agent.

35. The method or use defined in claim 34, wherein the antioxidant compound comprises sodium metabisulfite.

36. The method or use defined in any one of claims 34-35, wherein the chelating agent comprises ethylenedinitrotetraacetic acid.

37. The method or use defined in any one of claims 34-35, wherein the chelating agent comprises ethylenedinitrotetraacetic acid disodium salt.

38. The method or use defined in any one of claims 34-37, wherein the prolonged release compound is selected from the group consisting of a hydrophobic polymer, a hydrophilic polymer, a protein-derived material, a gum, a substituted or unsubstituted hydrocarbon, a digestible carbohydrate, a fatty acid, a fatty alcohol, a glyceryl ester of a fatty acid, a natural oil, a synthetic oil, a natural wax, a synthetic wax and any mixture of two or more of any of these.

39. The method or use defined in any one of claims 34-37, wherein the prolonged release compound is selected from the group consisting of a cellulose ether, an acrylic based polymer, an acrylic based copolymer, a methacrylic based polymer, a methacrylic based copolymer, a fatty alcohol and any mixture of two or more of any of these.

40. The method or use defined in any one of claims 34-37, wherein the prolonged release compound is selected from the group consisting of a neutral acrylic based polymer, a neutral acrylic based copolymer, a neutral methacrylic based polymer, a neutral methacrylic based copolymer, a hydrophobic cellulose ether, a fatty alcohol and any mixture of two or more of any of these.

41. The method or use defined in any one of claims 34-37, wherein the prolonged release compound is ethyl cellulose.

42. The method or use defined in any one of claims 34-41, wherein the granule is selected from an uncoated microcrystalline cellulose granule and a mannitol-polyvinylpyrrolidone granule.

43. The method or use defined in any one of claims 34-42, further comprising:
(d) a third layer coated on the second layer, the third layer comprising a moisture barrier agent.

44. The method or use defined in claim 43, wherein the moisture barrier agent comprises a polyvinyl alcohol-polyethylene glycol graft copolymer.

45. The method or use defined in any one of claims 34-44, wherein the prolonged release composition is in the form of a capsule.

46. The method or use defined in claim 45, wherein the capsule containing the plurality of coated beads.

47. The method or use defined in any one of claims 45-46, wherein the capsule is a hydroxypropyl methyl cellulose capsule.

48. The method or use defined in claim 33, wherein the prolonged release pharmaceutical composition comprises a prolonged release pharmaceutical dosage form comprising a plurality of coated beads disposed in a hydroxypropyl methyl cellulose capsule, each of the coated beads comprising:
(a) a granule;
(b) a first layer coated on the granule, the first layer comprising: (i) hydromorphone hydrochloride, (ii) naloxone hydrochloride, (iii) an antioxidant compound, and (iii) a chelating compound, wherein (i) and (ii) are present in a weight ratio of about 2:1;
(c) a second layer coated on the first layer, the second layer comprising ethyl cellulose; and
(d) a third layer coated on the second layer, the third layer comprising a polyvinyl alcohol-polyethylene glycol graft copolymer.

49. The method or use defined in claim 48, wherein the granule is an uncoated microcrystalline cellulose granule.

50. The method or use defined in claim 48, wherein the granule is a mannitol-polyvinylpyrrolidone granule.

51. The method or use defined in claim 33, wherein the prolonged release pharmaceutical composition comprises a prolonged release pharmaceutical dosage form in the form of a capsule containing coated beads derived from the following formulation:

| Ingredient | Amount per capsule (mg) |
|---|---|
| Hydromorphone hydrochloride | 3.00 |
| Naloxone hydrochloride dihydrate | 1.65 |
| Microcrystalline cellulose spheres | 44.72 |
| Hydroxypropyl methycellulose/ | 0.50 |
| polyethylene glycol | |
| Sodium metabisulfite | 0.05 |
| Disodium EDTA dihydrate | 0.05 |
| Aqueous ethylcellulose dispersion | 3.95 |
| Polyvinyl alcohol-polyethylene glycol | 1.41 |
| graft copolymer | |
| Water | q.s. (to 55 mg) |

52. The method or use defined in any one of claims 1-32, wherein the oral pharmaceutical composition immediate release pharmaceutical composition.

53. The method or use defined in claim 52, wherein the immediate release pharmaceutical composition comprises a diluent.

54. The method or use defined in any one of claims 52-53, wherein the immediate release pharmaceutical composition comprises a colourant.

55. The method or use defined in any one of claims 52-54, wherein the immediate release pharmaceutical composition comprises a lubricant.

56. The method or use defined in claim 52, wherein the immediate release pharmaceutical composition is derived from one of the following formulations:

| | Strength | | | |
|---|---|---|---|---|
| | 1/0.5 mg | 2/1 mg | 4/2 mg | 8/4 mg |
| Tablet Core Constituent | mg/tablet | mg/tablet | mg/tablet | mg/tablet |
| Hydromorphone | 1.00 | 2.00 | 4.00 | 8.00 |
| hydrochloride¹ | | | | |
| Naloxone hydrochloride | 0.55 | 1.10 | 2.20 | 4.40 |
| dihydrate² | | | | |
| Lactose anhydrous | 84.45 | 84.00 | 84.00 | 136.10 |
| DC Yellow #10 Lake | 0.30 | 0.40 | 0.30 | — |
| FD&C Blue #1 Lake | 0.04 | — | — | — |
| FD&C Red #30 Lake | — | 0.06 | — | — |
| Sodium stearyl fumarate | 1.36 | 1.34 | 1.40 | 2.30 |
| Total core | ~88 | ~89 | ~92 | ~151 |

¹calculated based on assay
²calculated based on assay and moisture content.

57. The method or use defined in any one of claims 1-56 while treating pain in the subject.
